(19) United States
(12) Patent Application Publication
(10) Pub. No.: US 2024/0382083 A1
(43) Pub. Date: Nov. 21, 2024

(54) Systems and Methods for Enabling Patients to Flexibly, Rapidly and Independently Access Eye Exams

(71) Applicant: Radius XR, LLC, Pleasanton, CA (US)

(72) Inventors: Chris Bradley, Baltimore, MD (US); Ammad Khan, Pleasanton, CA (US); John Trefethen, Pleasanton, CA (US)

(21) Appl. No.: 18/614,443

(22) Filed: Mar. 22, 2024

Related U.S. Application Data
(63) Continuation-in-part of application No. 17/456,490, filed on Nov. 24, 2021.
(60) Provisional application No. 63/492,197, filed on Mar. 24, 2023.
(60) Provisional application No. 63/223,005, filed on Jul. 18, 2021.
(60) Provisional application No. 63/118,538, filed on Nov. 25, 2020.

(51) Int. Cl. A61B 3/032; A61B 3/00; G16H 10/60; G16H 40/67

(52) U.S. Cl. CPC A61B 3/032; A61B 3/0008; G16H 10/60; G16H 40/67

(57) ABSTRACT

Systems and methods for delivering an eye exam order to a patient using a head-mounted vision device are provided. The head-mounted vision device includes a display in electrical and data communication with a computing device that is in data communication with at least one server over a network. The computing device executes a plurality of plurality of instructions for generating, for display on the computing device, a first graphical user interface to enable the patient to self-check-in for receiving the eye exam order; generating, for display on the head-mounted vision device, a first view of a second graphical user interface to deliver a predefined plurality of content items to the patient; and generating, for display on the computing device, data indicative of a second view of the second graphical user interface to display progress status of the predefined plurality of content items to a clinical staff person.

### CROSS-REFERENCE

The present specification relies on U.S. Provisional Patent Application No. 63/492,197, titled “Systems and Methods for Enabling Patients to Flexibly, Rapidly and Independently Access Eye Exams”, filed on Mar. 24, 2023, for priority, the entirety of which is herein incorporated by reference in its entirety.

The present specification is also a continuation-in-part application of U.S. patent application Ser. No. 17/456,490, titled “Methods and Systems for Evaluating Vision Acuity and/or Conducting Visual Field Tests in a Head-Mounted Vision Device” and filed on Nov. 24, 2021, which relies on U.S. Provisional Patent Application No. 63/223,005, titled “Methods and Systems for Determining a Variety of Vision Parameters in a Head-Mounted Vision Device” and filed on Jul. 18, 2021, and U.S. Provisional Patent Application No. 63/118,538, titled “Method and System for Determining Vision Acuity in a Head-Mounted Vision Device” and filed on Nov. 25, 2020, for priority, all of which are herein incorporated by reference in their entirety.

The present specification also relates to U.S. patent application Ser. No. 17/804,366, titled “Methods and Systems for Enabling the Remote Testing of Vision and Diagnosis of Vision-Related Issues” and filed on May 27, 2022, which is a continuation application of U.S. patent application Ser. No. 17/202,748, titled “Multi-Modal Vision Enhancement System”, filed on Mar. 16, 2021, and issued as U.S. Pat. No. 11,372,479 on Jun. 28, 2022, which, in turn, relies on U.S. Provisional Patent Application No. 62/990,422, of the same title and filed on Mar. 16, 2020, for priority.

The present specification also relates to U.S. patent application Ser. No. 17/643,414, titled “Method and System for Remote Clinician Management of Head-Mounted Vision Assist Devices” and filed on Dec. 8, 2021, which relies on U.S. Provisional Patent Application No. 63/223,005, titled “Methods and Systems for Determining a Variety of Vision Parameters in a Head-Mounted Vision Device” and filed on Jul. 18, 2021, and U.S. Provisional Patent Application No. 63/122,840, titled “Method and System for Remote Management of Head-Mounted Vision Assist Devices” and filed on Dec. 8, 2020, for priority.

The present specification also relates to U.S. patent application Ser. No. 17/445,935, titled “Clinician-Modifiable Portable User Vision Enhancement System” and filed on Aug. 25, 2021, which is a continuation application of U.S. patent application Ser. No. 16/447,481, titled “Methods and Systems for Generating a Magnification Region in Output Video Images”, filed on Jun. 20, 2019, and issued as U.S. Pat. No. 11,144,119 on Oct. 12, 2021, which is a continuation application of U.S. patent application Ser. No. 16/137,003, titled “Methods and Apparatus for Vision Enhancement”, filed on Sep. 20, 2018, and issued as U.S. Pat. No. 10,444,833 on Oct. 15, 2019, which is a continuation application of U.S. patent application Ser. No. 14/937,373, of the same title, filed on Nov. 10, 2015, and issued as U.S. Pat. No. 10,146,304 on Dec. 4, 2018, which, in turn, relies on U.S. Provisional Patent Application No. 62/077,434, titled “Low Vision Head-Mounted Visual Enhancement Device” and filed on Nov. 10, 2014, U.S. Provisional Patent Application No. 62/131,957, titled “Eye Tracking Systems” and filed on Mar. 12, 2015, and U.S. Provisional Patent Application No. 62/155,972, titled “Universal Testbed and Platform to Improve the Performance of Low Vision Patients” and filed on May 1, 2015, for priority.

The present specification also relates to U.S. patent application Ser. No. 17/931,219, titled “Methods and Apparatus for Contrast Sensitivity Compensation” and filed on Sep. 12, 2022, which is a continuation application of U.S. patent application Ser. No. 17/084,233, of the same title, filed on Oct. 29, 2020, and issued as U.S. Pat. No. 11,475,547 on Oct. 18, 2022, which is a continuation application of U.S. patent application Ser. No. 16/274,976, of the same title, filed on Feb. 13, 2019, and issued as U.S. Pat. No. 10,963,999 on Mar. 30, 2021, which, in turn, relies on U.S. Provisional Patent Application No. 62/629,774, titled “Methods and Apparatus for Low Vision Enhancement” and filed on Feb. 13, 2018, for priority.

The present specification also relates to U.S. patent application Ser. No. 18/057,928, titled “Methods and Apparatuses for Compensating for Retinitis Pigmentosa” and filed on Nov. 22, 2022, which is a continuation application of U.S. patent application Ser. No. 16/503,098, of the same title, filed on Jul. 3, 2019, and issued as U.S. Pat. No. 11,546,527 on Jan. 3, 2023, which, in turn, relies on U.S. Provisional Patent Application No. 62/697,173, titled “Compensating for Retinitis Pigmentosa: Using dynamic shifts in field of view and magnification” and filed on Jul. 5, 2018, for priority.

The present specification also relates to U.S. patent application Ser. No. 14/984,218, titled “Night Driving System and Method” and filed on Dec. 30, 2015, which relies on U.S. Provisional Patent Application No. 62/131,957, titled “Eye Tracking Systems” and filed on Mar. 12, 2015, for priority.

All of the above referenced applications are herein incorporated by reference in their entirety.

### FIELD

The present specification relates to vision assist and/or diagnostic systems and methods. Specifically, the embodiments disclosed herein describe use of a head-mounted vision device connected to and driven by a computing device that executes a software application to: a) enable patients to self-check-in for an eye exam, and b) enable patients to efficiently and independently access one or more eye exams that have been ordered by a clinician; and c) to enable clinicians to view a patient's visual field trends over a period of time.

### BACKGROUND

In a conventional clinical setup, visual acuity tests determine how well an individual can see a letter or symbol of a given size from a predefined distance. For example, in a Snellen test, letters are arranged in rows and/or columns. From row to row, the letters have different sizes, typically decreasing in size as one visually progresses from a higher row to a lower row. Standing 14 to 20 feet away, an individual attempts to identify each letter accurately. A value representative of the individual's visual acuity may be determined based on how far the individual is able to accurately progress in the chart (the smallest letter the individual is able to accurately identify). Alternatively, in a Random or Tumbling E test, an individual is presented a series of “E” images of different sizes and instructed to identify the direction each letter “E” is facing, such as up, down, left, or right. Again, a value representative of the individual's visual acuity may be determined based on how far the individual is able to accurately progress in the chart (the smallest letter the individual is able to accurately identify). Regardless of the type of visual acuity test being used, a conventional visual acuity test requires an individual to accurately identify the type of, or orientation of, differently sized letters, symbols, or figures, generally referred to as optotypes.

Also, conventionally, testing a user's visual field and/or peripheral vision is performed with dedicated devices and systems. Two common vision tests are the Humphrey visual field test (VFT) and the Amsler grid test. Other visual field tests may include a confrontation visual field test, an automated static perimetry test, a kinetic visual field test, a frequency doubling perimetry test, and an electroretinography (ERG) test. For the automated static perimetry test, a user is provided with a device that displays visual stimuli (typically a small spot of light) at a predetermined set of test locations in the user's peripheral visual field. The user is instructed to fixate at the center of the visual field and to detect any spot of light that appears in the peripheral visual field. Typically, the spot of light is presented at each test location multiple times, and at different contrast levels. The contrast level at which the probability of detection is at a predetermined level (usually set to 50% probability) represents the contrast threshold at that test location. If the contrast threshold is sufficiently high (equivalently, contrast sensitivity is sufficiently low), the system determines that the user may have a deficit in his or her visual field at that test location. Stimulus presentation at any test location may be optimized to reduce the number of measurements by leveraging estimates of contrast thresholds at neighboring test locations, dynamically changing the time between stimulus presentations based on the distribution of user response times and leveraging normative databases of contrast sensitivity for different age groups. Examples of algorithms that implement such optimization procedures are SITA, ZEST and SWeLZ. In certain cases, the VFT may be used to present the stimulus at only the highest possible contrast level as a fast screening method.

The Amsler Grid test is a tool for discovering a user's visual impairments and entails a user looking at a printed grid of squares to determine whether there are any perceived artifacts or irregularities in the grid. The grid structure used for the test is simple and the contents within the grid are usually known to the user, which makes it easier to observe and note any differences between the actual content and what is visually perceived. Describing abnormal vision can be done on paper with a similar grid where a user can mark the areas of distortion for different kinds of distortions that may occur. Amsler grids are commonly 20×20 squares that may be placed at a distance to a user's eye such that each square subtends one degree of visual angle. Different versions of the grid may have varying features, such as color, for example. The Amsler Grid test is subjective; thus, a severity of vision impairment cannot easily be quantified through the test. The test is intended to be a quick and simple screening tool to determine common vision abnormalities such as, but not limited to scotoma, voids, holes, blind spots, and missing area in a user's vison.

Since vision tests (both field and acuity) are conducted using different systems and settings, the test application and processes vary between the systems and vary between physicians applying the tests and vary over time, making the test results less comparable and less precise due to introduction of these multiple variations. There is thus a need for a controlled environment for conducting these vision tests, such that the influence of the variations is minimized. Additionally, the outputs of the vision tests are typically stored in paper format or in a digital format in a manner that fails to aggregate, integrate, or cross-correlate the results of the tests, making comparisons between tests difficult and impractical.

In some configurations, the controlled environment for conducting these vision tests is provided using a head-mounted device that may operate in a diagnostic mode in which a wearer's degree of visual acuity may be assessed. In particular, the head-mounted device may be configured to execute a plurality of visual tests, elicit responses from the wearer which are input into the device, and, based on those responses, determine a value indicative of the wearer's visual acuity. Also, clinicians need to be efficient and service as many patients as possible with the least amount of staff.

Accordingly, there is a need for a clinical setup that provides systems and methods for vision testing that enable reduced variance and provide improved diagnostic results allowing for comparison between patients and over time. There is also a need for systems and methods that provide a streamlined workflow for enabling a patient to self-check-in for one or more eye exams and efficiently as well as independently access one or more eye exams, for enabling a skeletal staff for creating eye exam orders, scheduling the eye exam orders and inputting patient information, and for enabling a clinician to access and analyze diagnostic results.

### SUMMARY

The following embodiments and aspects thereof are described and illustrated in conjunction with systems, tools and methods, which are meant to be exemplary and illustrative, and not limiting in scope. The present application discloses numerous embodiments.

In some embodiments, the present specification describes a method of delivering an eye exam order to a patient using a head-mounted vision device configured to be positioned on the patient's head, wherein the head-mounted vision device includes a display in electrical and data communication with a first computing device that is in data communication with at least one server over a network, and wherein the first computing device includes a non-transient memory in data communication with at least one processor and adapted to store programmatic instructions that, when executed, execute said method, the method comprising: generating, for display on the first computing device, data indicative of a first graphical user interface to enable the patient to self-check-in for receiving the eye exam order; positioning the head-mounted vision device on the patient's head; generating, for display on the head-mounted device, data indicative of a first view of a second graphical user interface to deliver a predefined plurality of content items to the patient; and generating, for display on the first computing device, data indicative of a second view of the second graphical user interface to display progress status of the predefined plurality of content items to a clinical staff person.

Optionally, the first graphical user interface is configured to display the patient's information and eye exam order amongst a plurality of patients' information and associated eye exam orders.

Optionally, the patient, using the first computing device, actuates the eye exam order to self-check-in.

Optionally, the second graphical user interface includes a visual graphical element having first and second toggle positions. Optionally, enabling the first toggle position causes the first view to be displayed, and wherein enabling the second toggle position causes the second view to be displayed.

Optionally, the predefined plurality of content items comprises one or more eye exams and one or more educational content related to one or more eye ailments.

Optionally, the method further comprises generating, for display on the first computing device, data indicative of a third graphical user interface to enable the clinical staff person to add at least one of: one or more eye exam templates, one or more eye exams and one or more educational content to the eye exam order.

Optionally, the method further comprises generating, for display on a second computing device, data indicative of a fourth graphical user interface to enable a clinician to select results of two or more eye exams associated with one or more eye exam orders delivered at different points in time to the patient, wherein the selected two or more eye exams may or may not correspond to a same eye exam order, wherein the second computing device is in data communication with the at least one server and the first computing device over the network, and wherein the at least one server stores said results of the two or more eye exams; and generating, for display on the second computing device, data indicative of a fifth graphical user interface configured to display an analysis of said results of the two or more eye exams.

Optionally, the results of the two or more eye exams are generated by a) obtaining a first value at each first stimulus location in the first of the two or more eye exams, b) obtaining a second value at each second stimulus location in the second of the two or more eye exams, c) if the first stimulus location and the second stimulus location are equivalent, applying a function to generate a third value for said equivalent location and displaying said third value in the fifth graphical user interface and d) if the first stimulus location has no equivalent location in the second of the two or more eye exams and/or if the second stimulus location has no equivalent location in the first of the two or more eye exams, displaying said first value and/or said second value in the fifth graphical user interface.

Optionally, the fifth graphical user interface is configured to display the analysis of said results of the two or more eye exams side-by-side for comparison.

Optionally, the analysis corresponds to determining a change in one or more parameters of said results and wherein the fifth graphical user interface is configured to display the change.

In some embodiments, the present specification describes a computer readable program adapted to deliver an eye exam order to a patient using a head-mounted vision device positioned on the patient's head, wherein the head-mounted vision device includes a display in electrical and data communication with a first computing device that is in data communication with at least one server over a network, wherein the first computing device includes at least one processor in data communication with a non-transient memory that stores the computer readable program, and wherein the computer readable program comprises a plurality of programmatic instructions that, when executed by the at least one processor: generates, for display on the first computing device, data indicative of a first graphical user interface to enable the patient to self-check-in for receiving the eye exam order; generates, for display on the head-mounted device, data indicative of a first view of a second graphical user interface to deliver a predefined plurality of content items to the patient; and generates, for display on the first computing device, data indicative of a second view of the second graphical user interface to display progress status of the predefined plurality of content items to a clinical staff person.

Optionally, the first graphical user interface is configured to display the patient's information and eye exam order amongst a plurality of patients' information and associated eye exam orders.

Optionally, the patient, using the first computing device, actuates the eye exam order to self-check-in.

Optionally, the second graphical user interface includes a visual graphical element having first and second toggle positions. Optionally, enabling the first toggle position causes the first view to be displayed, and wherein enabling the second toggle position causes the second view to be displayed.

Optionally, the predefined plurality of content items comprises one or more eye exams and one or more educational content related to one or more eye ailments.

Optionally, the computer readable program is further adapted to generate, for display on the first computing device, data indicative of a third graphical user interface to enable the clinical staff person to add at least one of: one or more eye exam templates, one or more eye exams and one or more educational content to the eye exam order.

Optionally, the computer readable program is further adapted to generate, for display on a second computing device, data indicative of a fourth graphical user interface to enable a clinician to select results of at least two eye exams associated with one or more eye exam orders delivered at different points in time to the patient, wherein the selected at least two eye exams may or may not correspond to a same eye exam order, wherein the second computing device is in data communication with the at least one server and the first computing device over the network, and wherein the at least one server stores said results of the at least two eye exams; and generate, for display on the second computing device, data indicative of a fifth graphical user interface displaying an analysis of said results of the at least two eye exams.

Optionally, the fifth graphical user interface is configured to display the analysis of said results of the two or more eye exams side-by-side for comparison.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is a block diagram showing a clinical setup of a vision assist and/or diagnostic system, in accordance with some embodiments of the present specification;

FIG. 2 is a flowchart of a plurality of exemplary steps of a method of delivering an eye exam order to a patient, in accordance with some embodiments of the present specification;

FIG. 3A is a first GUI generated by a VAD App, in accordance with some embodiments of the present specification;

FIG. 3B is a second GUI generated by the VAD App, in accordance with some embodiments of the present specification;

FIG. 3C represent first and second views of a third GUI generated by the VAD App, in accordance with some embodiments of the present specification;

FIG. 3D represents first, second, third, fourth and fifth views of a fourth GUI generated by the VAD App, in accordance with some embodiments of the present specification;

FIG. 3E is a fifth GUI generated by the VAD App, in accordance with some embodiments of the present specification;

FIG. 3F represents first and second views of a sixth GUI generated by the VAD App, in accordance with some embodiments of the present specification;

FIG. 4 is a flowchart of a plurality of exemplary steps of a method of enabling a clinician to analyze and view results of a patient's one or more eye exams, in accordance with some embodiments of the present specification;

FIG. 5A represents a first view of a seventh GUI generated by the VAD App, in accordance with some embodiments of the present specification;

FIG. 5B represents a second view of the seventh GUI generated by the VAD App, in accordance with some embodiments of the present specification;

FIG. 5C is an eighth GUI generated by the VAD App, in accordance with some embodiments of the present specification;

FIG. 6A is an alternate view of the seventh GUI generated by the VAD App, shown in FIGS. 5A and 5B, in accordance with alternate embodiments of the present specification;

FIG. 6B is an alternate view of the eighth GUI generated by the VAD App, shown in FIG. 5C, in accordance with alternate embodiments of the present specification;

FIG. 7A is a first view of a ninth GUI generated by the VAD App presented to enable selecting baseline and post-baseline eye exam data for merging, in accordance with alternate embodiments of the present specification;

FIG. 7B is a second view of the ninth GUI, in accordance with alternate embodiments of the present specification;

FIG. 8 is a tenth GUI generated by the VAD App presented for enabling creation of a custom eye exam order for a patient, in accordance with some embodiments of the present specification; and

FIG. 9 shows merging of standard eye exam data with custom eye exam data to generate merged data, in accordance with some embodiments of the present specification.

### DETAILED DESCRIPTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In various embodiments, a computing device includes an input/output controller, at least one communications interface and system memory. The system memory includes at least one random access memory (RAM) and at least one read-only memory (ROM). These elements are in communication with a central processing unit (CPU) to enable operation of the computing device. In various embodiments, the computing device may be a conventional standalone computer or alternatively, the functions of the computing device may be distributed across multiple computer systems and architectures.

In some embodiments, execution of a plurality of sequences of programmatic instructions or code enable or cause the CPU of the computing device to perform various functions and processes. In alternate embodiments, hard-wired circuitry may be used in place of, or in combination with, software instructions for implementation of the processes of systems and methods described in this application. Thus, the systems and methods described are not limited to any specific combination of hardware and software.

The term “module”, “application” or “engine” used in this disclosure may refer to computer logic utilized to provide a desired functionality, service or operation by programming or controlling a general purpose processor. Stated differently, in some embodiments, a module, application or engine implements a plurality of instructions or programmatic code to cause a general purpose processor to perform one or more functions. In various embodiments, a module, application or engine can be implemented in hardware, firmware, software or any combination thereof. The module, application or engine may be interchangeably used with unit, logic, logical block, component, or circuit, for example. The module, application or engine may be the minimum unit, or part thereof, which performs one or more particular functions.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

As used herein, the term “isopter” refers to a contour of similar visual sensitivity estimates. It is similar to a topographical map where there are contours representing similar elevations. Thus, in technical terms, an isopter is a contour line in a representation of the visual filed around the points representing the macula that passes through the points of equal visual acuity.

In embodiments of the present specification, it should be noted that while threshold and sensitivity refer to the same data that is being generated, they are inverses of each other (sensitivity=1/threshold).

### Overview

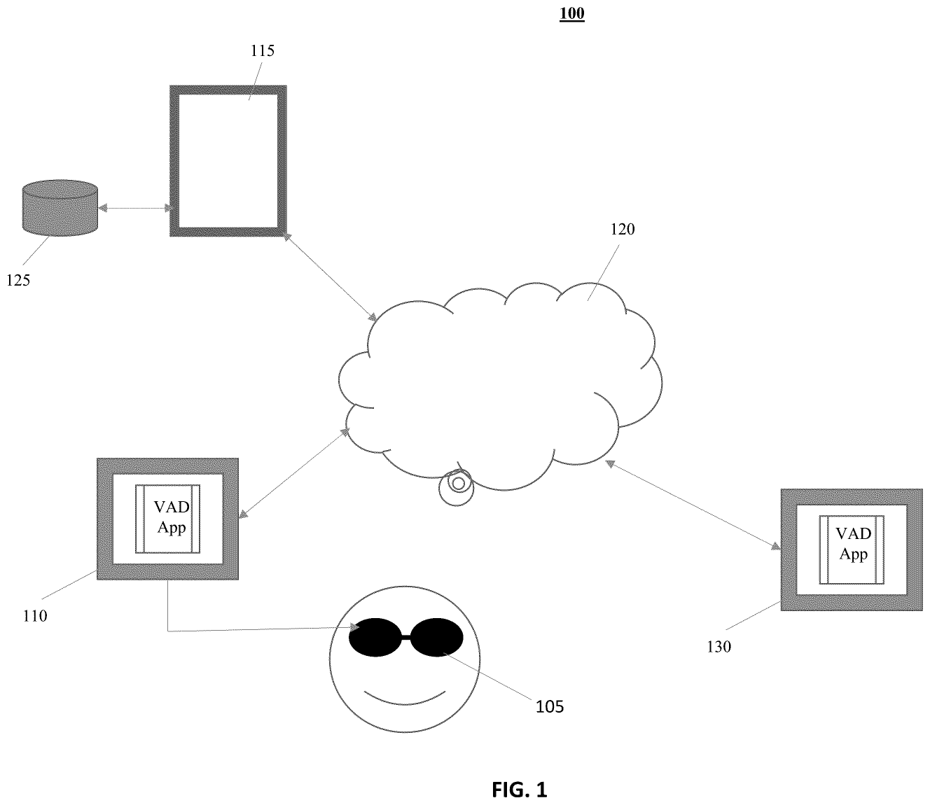

FIG. 1 is block diagram showing a clinical setup of a vision assist and/or diagnostic system 100, in accordance with some embodiments of the present specification. The system 100 includes a head-mounted vision device 105 which, when worn or mounted on the head of a patient, is in electrical and data communication with at least one co-located first computing device 110. In some embodiments, the at least one first computing device 110 is in data communication with at least one server 115 over a network 120 that may be wired and/or wireless. In some embodiments, the at least one server 115 includes or is in communication with a database system 125. The database system 125 stores a plurality of data corresponding to patients, eye exam orders, eye exams and results of the eye exams. In some embodiments, the at least one server 115 may be implemented by a cloud of computing platforms operating together as the at least one server 115.

In some embodiments, at least one second computing device 130, is also in data communication either directly with the at least one first computing device 110 and/or is in data communication with the at least one first computing device 110 through the network 120. In some embodiments, the at least one second computing device 130 may be located remotely from the at least one first computing device 110 and the head-mounted vision device 105. Alternatively, the at least one second computing device 130 may be co-located with the at least one first computing device 110 and the head-mounted vision device 105.

In some embodiments, the at least one first computing device 110 is provided with programming through a vision assist and diagnostic application, module or engine (hereinafter referred to as a “VAD App”). The VAD App implements a plurality of instructions or programmatic code to configure the at least one first computing device 110 to function: a) as a patient check-in device, b) as a technician or clinical staff person's device for inputting or adding eye exam orders, inputting or adding patient information, inputting or adding eye exam order templates and inputting or adding educational content, and c) as a patient testing or diagnostic device. When the at least one first computing device 110 is connected (that is, in electrical and data communication) to the head-mounted vision device 105, the least one computing device 110 enters into a patient testing mode and drives the head-mounted vision device 105 to deliver an eye exam order comprising a plurality of predefined sequential series of content or agenda.

In some embodiments, the at least one second computing device 130 may also implement the VAD App which may be used by the clinician to access results and various analyses of the patient's eye exams. In some embodiments, the VAD App may be implemented on the at least one server 115 and provided as a service to the at least one first computing device 110 and/or the at least one second computing device 130.

In some embodiments, the head-mounted vision device 105 is a passive display device and does not have its own processor or power source. In some embodiments, the head-mounted vision device 105 has at least one screen (or alternatively, left and right screens corresponding to a patient's left and right eyes) that displays, to the patient, the plurality of predefined sequential series of content or agenda associated with the eye exam order.

In various embodiments, the at least one first computing device 110 and at least one second computing device 130 comprise devices such as, but not limited to, personal or desktop computers, laptops, Netbooks, handheld devices such as smartphones, tablets, and PDAs and/or any other computing platform known to persons of ordinary skill in the art.

### Eye Exam Order Delivery Method

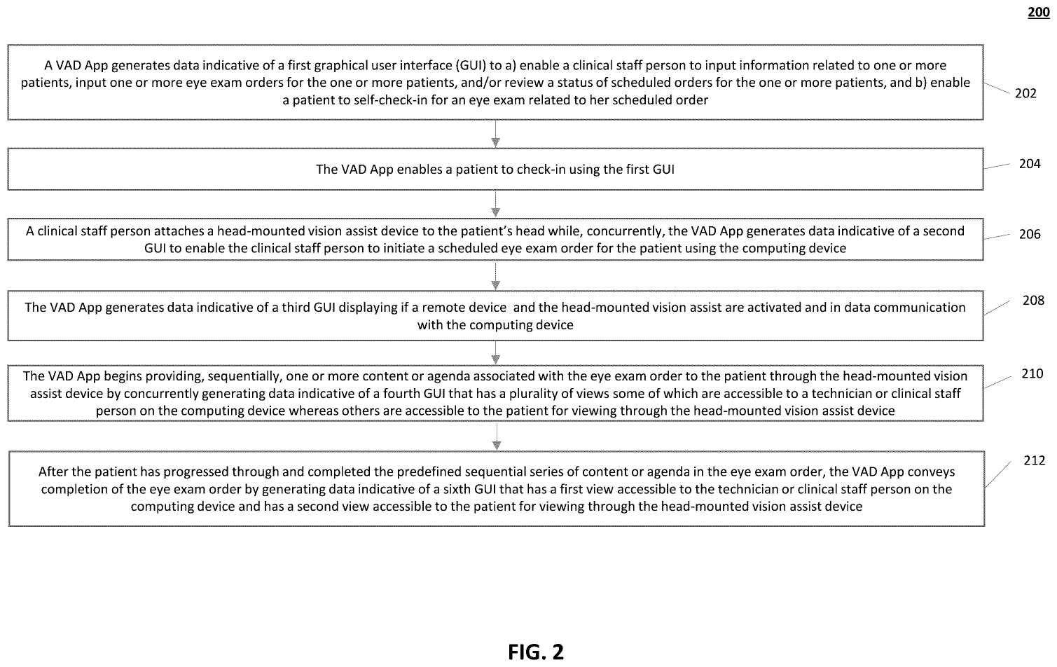

FIG. 2 is a flowchart of a plurality of exemplary steps of a method 200 of delivering an eye exam order to a patient, in accordance with some embodiments of the present specification. In embodiments, the method 200 is implemented by the vision assist and diagnostic application (“VAD App”) on the first computing device 110 in data communication with the head-mounted vision device 105. In various embodiments, the graphical user interfaces generated during the execution of any of the methods described herein may be available for display on a patient device or tablet (first computing device), on the head-mounted device, on the clinician's computing device (second computing device) or any combination thereof. In some embodiments, the first computing device 110 serves as both the patient device and clinician device. In some embodiments, the clinician and patient toggle between use of the computing device. In some embodiments, the patient and clinician have their own respective devices. It should be noted herein that the functionalities and graphical user interfaces described throughout this specification are configured such that they allow for the computing device to be used by both patient and clinician or for each of the patient and clinician to have their own dedicated computing devices. Further, a plurality of patient devices may be configured to communicate with one clinician device. Still further, a plurality of patient devices may be configured to communicate with a plurality of clinician devices. In the examples below, the clinician is a primary user of computing device 110 while the patient is the primary user of head-mounted device 105.

Referring now to FIGS. 1, 2 and 3A through 3F simultaneously, at step 202, the VAD App generates data indicative of a first graphical user interface (GUI) 300a to a) enable a clinical staff person to input information related to one or more patients, input one or more eye exam orders for the one or more patients, and/or review a status of scheduled orders for the one or more patients, and b) enable a patient to self-check-in for an eye exam related to her scheduled order.

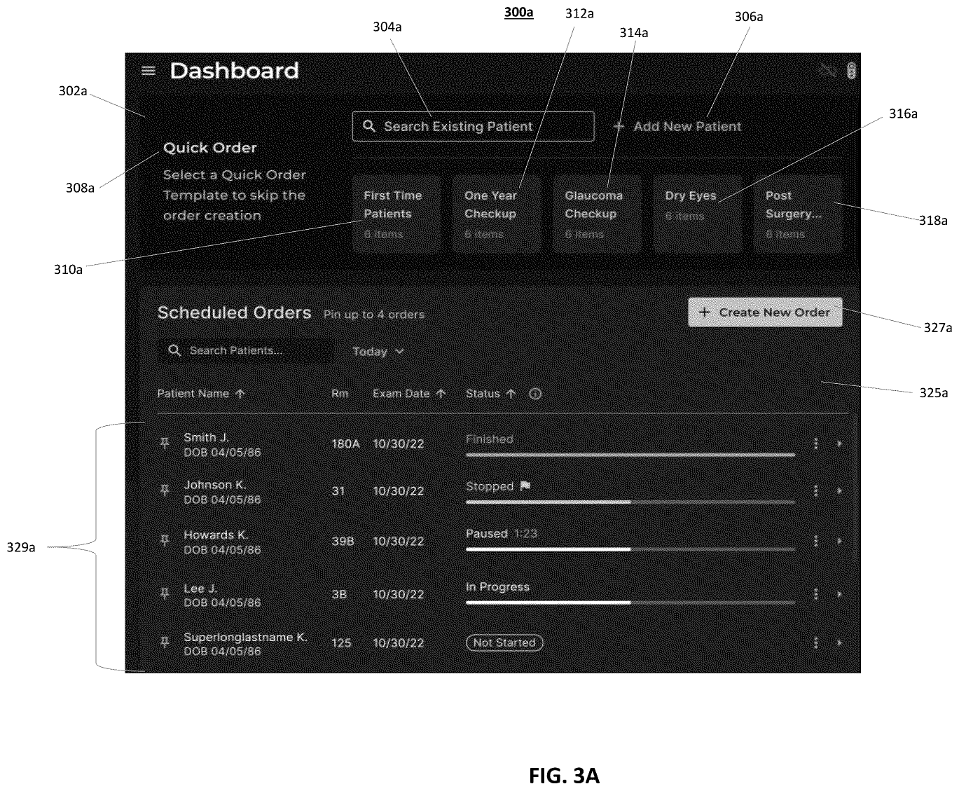

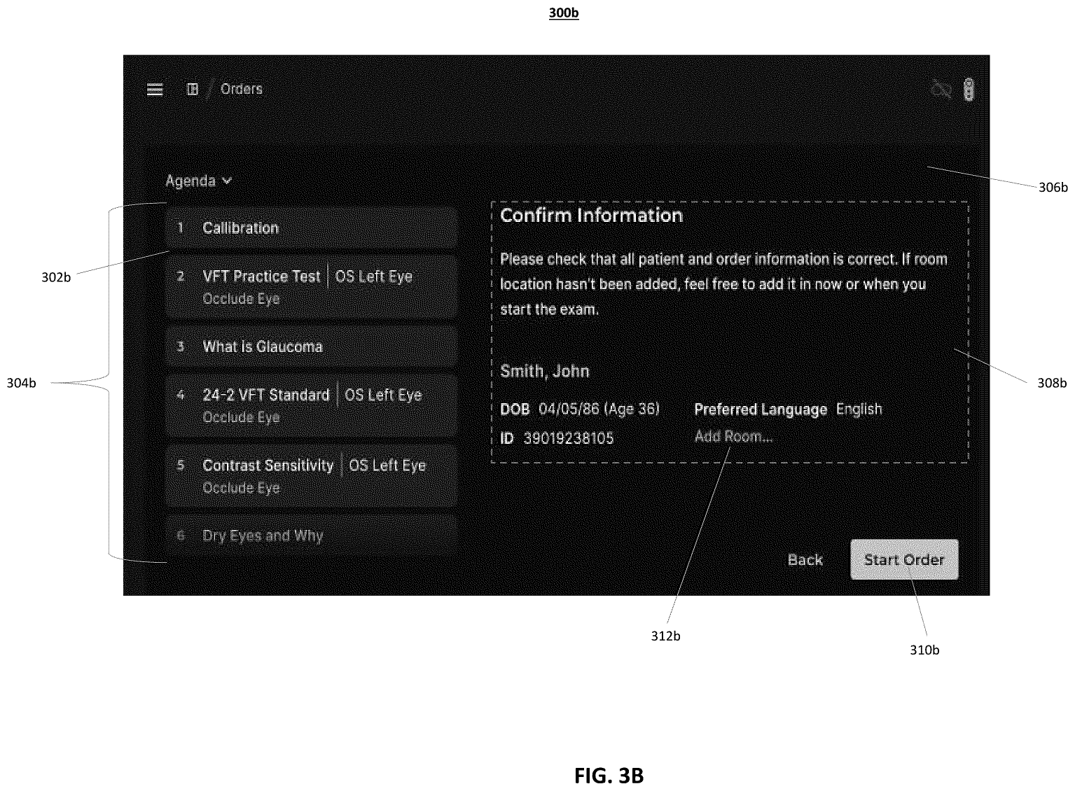

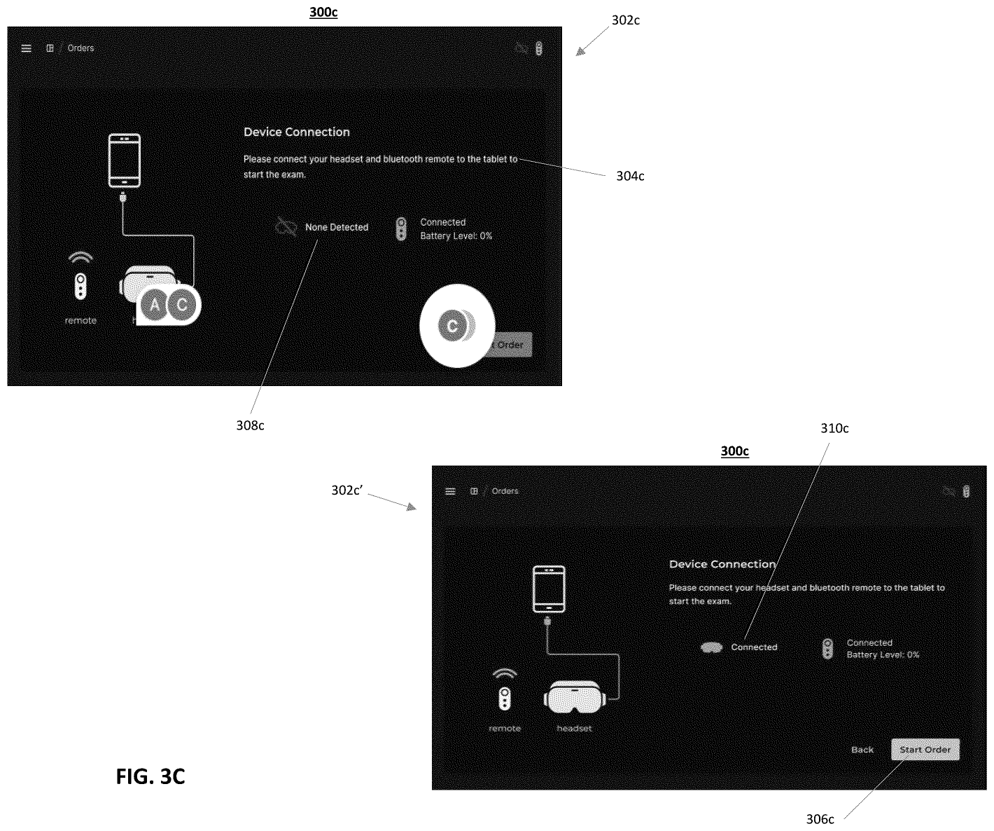

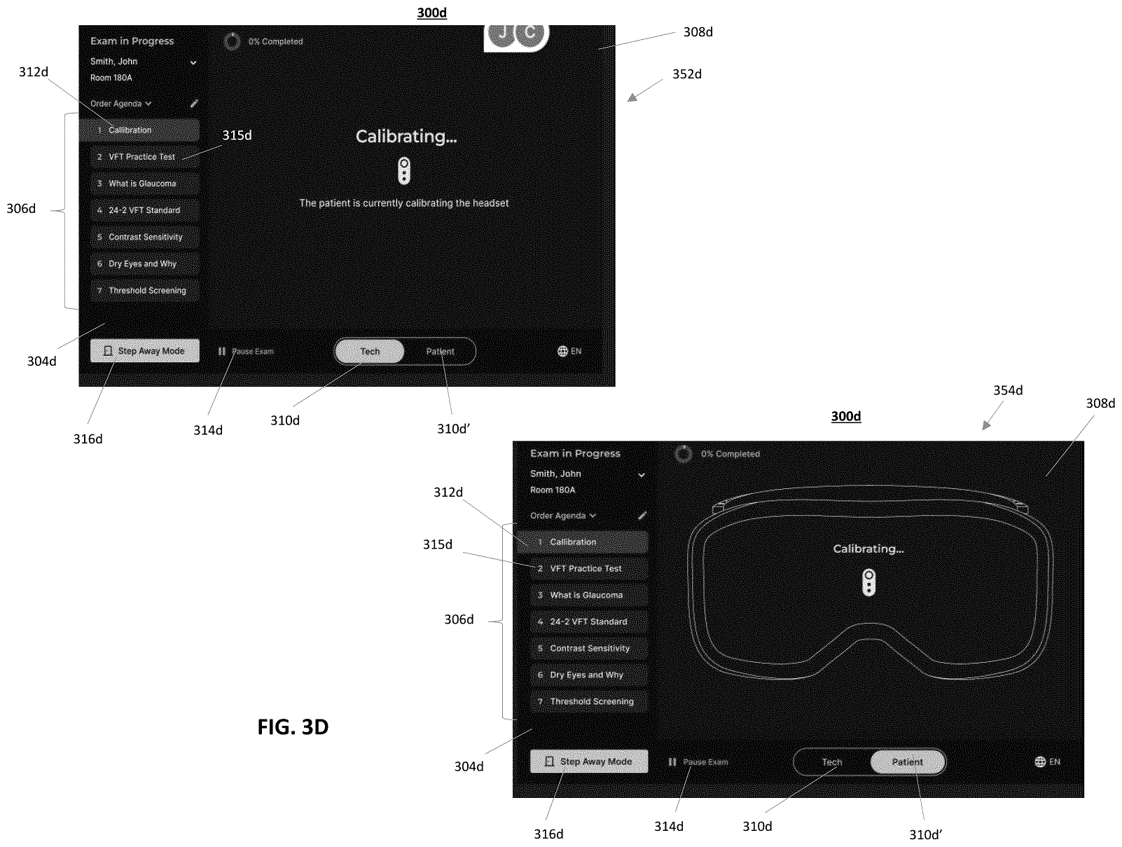

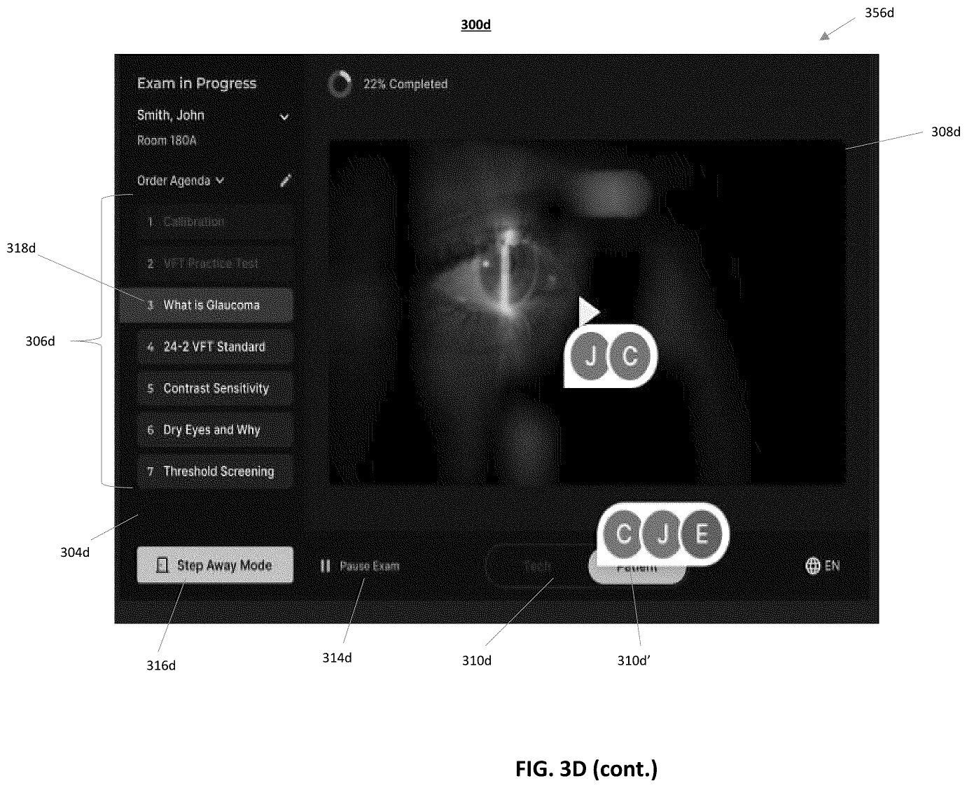

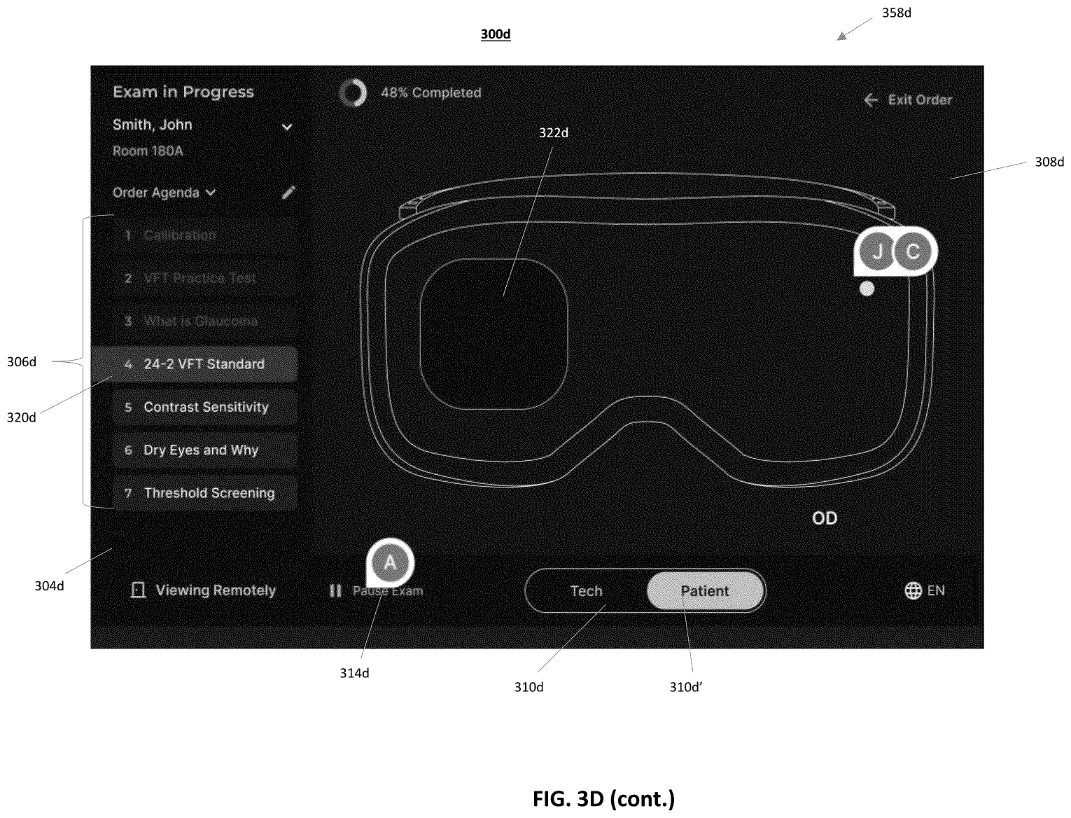

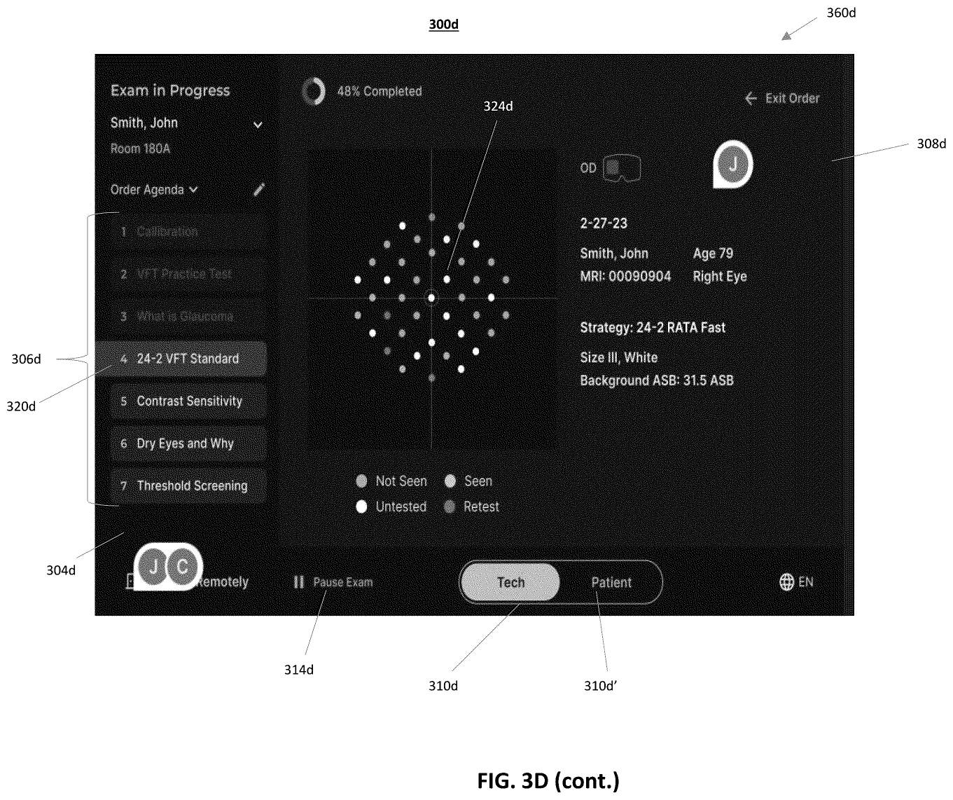

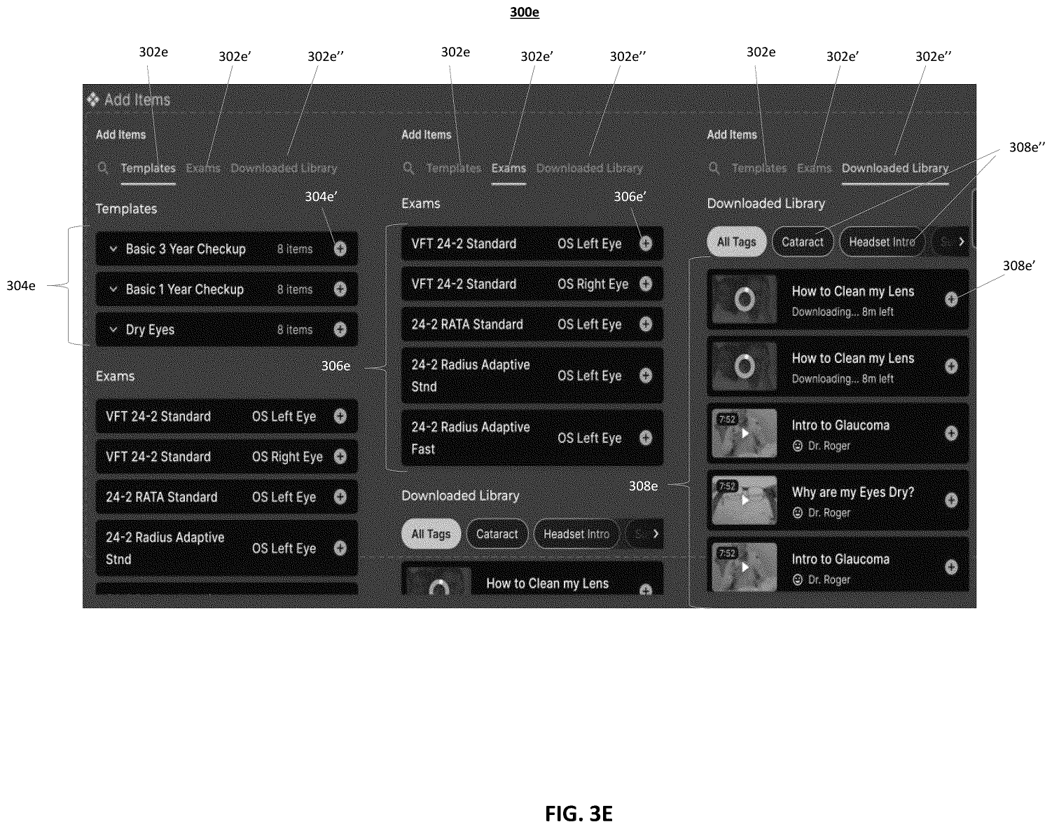

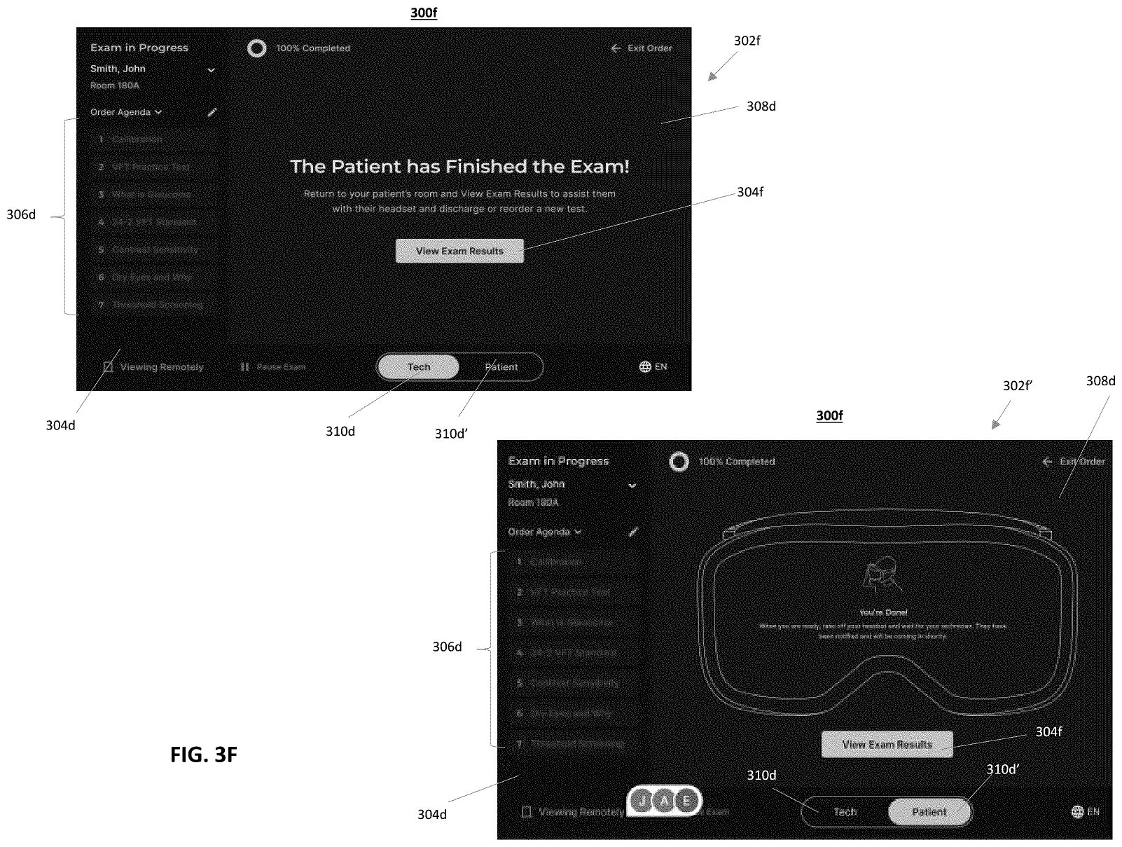

FIG. 3A shows the first GUI 300a generated by the VAD App, in accordance with some embodiments of the present specification. The first GUI 300a is configured as a dashboard that enables a clinical staff person to perform a plurality of tasks while also enabling a patient to self-check-in for an eye exam related to her scheduled order. As shown, the first GUI 300a has a first portion or area 302a including a text box 304a to allow searching information and eye exam orders related to an existing patient. A visual graphical element 306a (titled “add new patient”) when actuated (for example, by touch-based clicking) allows inputting information related to a new patient which is then added to the database 125. Another visual graphical element 308a (titled “quick order”) when actuated allows selecting an order template, from a plurality of pre-stored or pre-configured templates related to a plurality of eye ailments and diagnostics, for a patient. The selected order template is then automatically stored in the database 125 in association with the patient (such as, for example, in association with a unique ID of the patient in the system).

In some embodiments, the first portion or area 302a includes additional visual graphical elements such as a visual graphical element 310a which when actuated allows viewing information related to first time patients, another visual graphical element 312a which when actuated allows viewing related to patients characterized by one year checkup, another visual graphical element 314a which when actuated allows viewing information of patients and orders related to glaucoma ailments, another visual graphical element 316a which when actuated allows viewing information of patients and orders related to dry eye ailments, and yet another visual graphical element 318a which when actuated allows viewing information of patients and orders related to post surgery exams.

A second portion or area 325a allows viewing of a plurality of stored scheduled orders for patients and/or for creating a new eye exam order for a patient. A visual graphical element 327a (titled “create new order”) when actuated allows creation of a new eye exam order for a patient based on default eye exam order fields, patterns, and attributes corresponding to an eye exam. Thus, an order can be created and saved in the system by either selecting a built-in order template (using the visual graphical element 308a) or by actuating the visual graphical element 327a. As a non-limiting example, the second portion or area 325a shows a list 329a of stored scheduled orders for a day along with a plurality of attributes (associated with each of the scheduled orders) such as, for example, patient name, exam room, eye exam date and status.

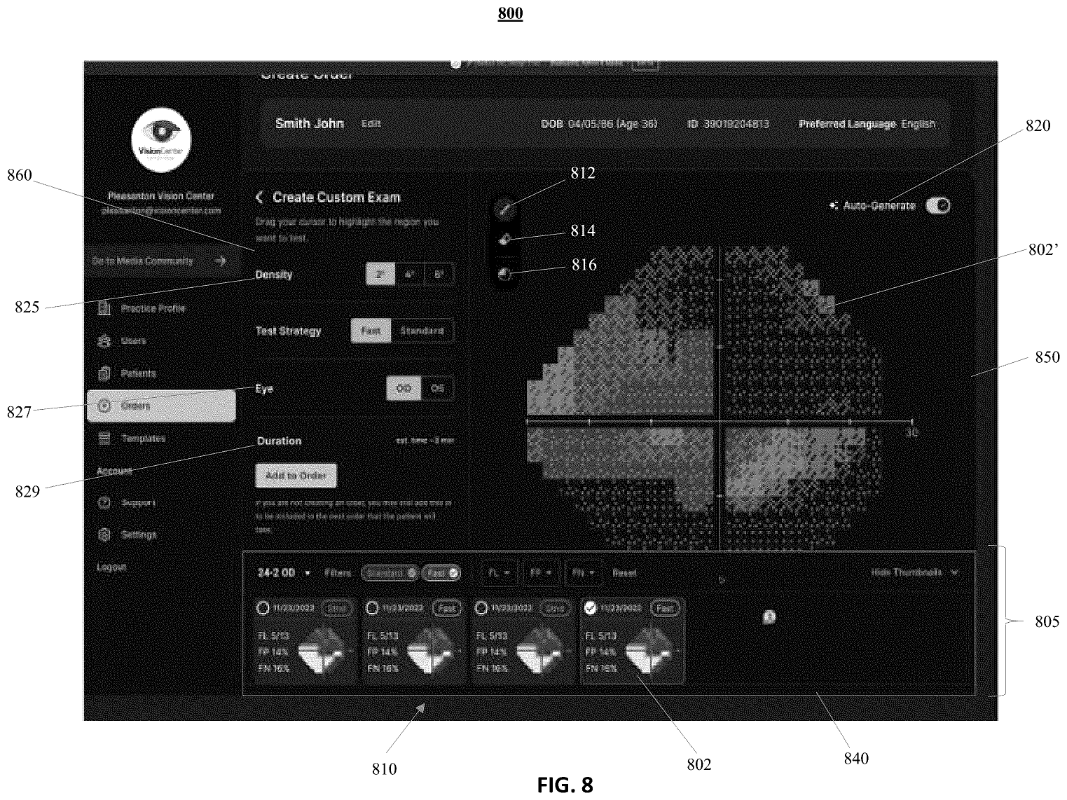

In some embodiments, the VAD App is configured to enable a clinical staff person to create a custom eye exam order for a patient and to give the clinical staff person control of regions for testing and clustering. FIG. 8 shows a GUI 800 generated and presented by the VAD App for enabling creation of a custom eye exam order for a patient, in accordance with some embodiments of the present specification. It should be appreciated that an objective of the GUI 800 is to empower a clinician to custom design (or select a designed eye exam from an auto-populated list) eye exam for a patient subject to certain constraints such as, for example, billable requirements, maximum test time or duration, and historical and/or real-time patient-specific eye exam results. In embodiments, the “billable” requirements refer to Current Procedural Terminology (“CPT”) codes. More specifically, CPT code 92083 (a medical procedure code) specifies a set of conditions that a visual field test must satisfy in order for it to be “billable”. The key requirements (which may be subject to change by the governing authority) are as follows:
i) It is a “threshold test”, which means that “thresholds” or sensitivity is estimated. For example, for an automated static perimetry test, the intensity of the stimulus at which the observer detects the stimulus 50% of the time is estimated.
ii) The visual field test covers three isopters. An isopter refers to a contour of similar sensitivity. Typical visual field test patterns have test points that cover at least three contours of sensitivity values, with one within the other (similar to a topographical map).

In embodiments, the clinical staff person may select a custom region to test based on a previous visual field test as the previous visual field test may help guide which regions to test with greater density. The results of the custom test and the previous visual field might, in some cases, be merged afterwards (as explained in various parts of the specification) to provide a map with a greater density of test points in regions of greater interest, which are typically the boundaries of scotomas. In an alternative embodiment, testing is dynamic—that is, the test may begin with a known test pattern (such as 24-2) but as the testing progresses, the algorithm is configured and designed to determine whether more testing is needed in certain regions and whether less testing is needed in other regions.

In some embodiments, the clinical staff person may choose a previously administered/conducted or a real-time eye exam (based on a predefined standard eye exam), such as eye exam 802, from a horizontal tray 805 configured to display a plurality of eye exams 810 displayed in visual miniature forms/icons. The tray 805 is positioned at a bottom portion 840 of the GUI 800. Selecting the eye exam 802 causes data 802′ indicative of the eye exam 802 to be displayed in a first window 850 positioned above the tray 805.

Subsequently, in some embodiments, the clinical staff person may use at least one of a plurality of drawing tools to define and/or manipulate areas of interest within the displayed data 802′. In embodiments, the plurality of drawing tools includes a first visual graphical element 812 indicative of a brush tool, a second visual graphical element 814 indicative of an eraser tool, and a third visual graphical element 816 indicative of a quadrant or lasso tool. Each of the plurality of drawing tools is configured to enable the clinical staff person to define and/or manipulate one or more test points or locations within the displayed data 802′. The defined and/or manipulated one or more test points or locations are visually highlighted or painted within the displayed data 802′. For example, for a patient already having hemi-vision loss (hemianopia), the clinical staff person can choose to test only the portions (hemi-region) where the patient still has vision in order to reduce test time.

Alternatively, in some embodiments, the clinical staff person may actuate a fourth visual graphical element 820 (titled ‘auto-generate’) which, when actuated, is configured to cause the VAD App to automatically generate a recommended custom eye exam, based on the selected eye exam 802, that may increase test points or locations in areas indicative of poor test results and/or may decrease test points or locations in areas indicative of normal visual field. The auto-generate functionality is configured such that it defines or paints the area(s), within the displayed data 802′, that should be tested more deeply (or more quickly). In some embodiments, the clinical staff person may further manipulate the area(s) (defined by the auto-generate functionality) using the plurality of drawing tools.

Referring back to FIG. 8, a second window 860, positioned above the tray 805 and adjacent the first window 850, displays a fifth visual graphical element 825 indicative of a density of test points or locations in areas of interest or concern (and that toggles among, for example, three density options for selection), a sixth visual graphical element 827 indicative of a left or right eye (and that toggles between left or right eye options for selection) and a seventh visual graphical element 829 indicative of a test time or duration of the custom eye exam, automatically estimated by the VAD App, based on the density option chosen. Thus, in embodiments, density affects test time or duration—when a higher density option is chosen for an area of interest or concern the VAD App provides an estimated higher test time or duration compared to an estimated lower test time or duration when a lower density option is chosen.

As described earlier, new or custom test points or locations are defined as a result of the activation of the auto-generate functionality (by actuating the fourth visual graphical element 820) and/or as a result of the clinical staff person using the plurality of drawing tools within the displayed data 802′. In some embodiments, creation of the new or custom test points or locations (of a custom eye exam) are subject to a plurality of filters or constraints. In some embodiments, the plurality of filters or constraints include billable requirements (that is, for example, must have three isopters and must be billable, as described above), maximum test time or duration allowed, and historical and/or real-time patient-specific eye exam results. In some embodiments, the VAD App is configured to automatically “gray-out” certain selections if choosing them will violate one or more filters or constraints (such as, for example, if selecting a density option will cause the test time or duration to be too large). In some embodiments, the options for modifying test time may include density (for example 2-degree, 4-degree, 6-degree density grids) and/or test strategy (for example, standard or fast) once a custom region is either manually selected (using the brush-like tool) or automatically selected using an algorithm.

In some embodiments, new or custom test points or locations are defined and/or manipulated based on data indicative of a real-time eye exam—that is, areas around good points or locations (where vision is not impaired) will be less densely tested than areas around points or locations where vision is impaired or visual defects are identified.

At step 204, the VAD App enables a patient to check-in using the first GUI 300a. In accordance with an objective of the present specification, the VAP App allows the patient to independently self-check-in using the computing device 110. In some embodiments, to do so, the patient clicks on his name from the list 329a of scheduled orders appearing for the day in the first GUI 300a. It should be appreciated that enabling the patient to self-check-in and initiate the exam order is desirable since clinicians need to be efficient and service as many patients as possible with the least amount of staff personnel.

At step 206, subsequent to the patient's check-in, the clinical staff person attaches the head-mounted vision device 105 to the patient's head while, concurrently, the VAD App generates data indicative of a second GUI 300b to enable the clinical staff person to initiate a scheduled eye exam order for the patient using the computing device 110. In some embodiments, attaching the head-mounted vision device 105 to the patient's head automatically places or configures the computing device 110 into a mode of delivering the eye exam order.

FIG. 3B shows the second GUI 300b generated by the VAD App, in accordance with some embodiments of the present specification. As shown, the second GUI 300b includes a first portion or area 302b that lists a predefined sequential series of content or agenda items 304b related to the eye exam order scheduled for the patient. A second portion or area 306b prompts the patient to check for his personal and order information provided in a third portion or area 308b. The third portion or area 308b also includes a link 312b to enable the patient to select a room in the clinic, if not already selected. A visual graphical element 310b (titled “start order”) when actuated causes the patient's eye exam order to be initiated.

At step 208, the VAD App generates data indicative of a third GUI 300c displaying if a remote device, such as the at least one second computing device, and the head-mounted vision assist are activated and in data communication with the computing device 110.

FIG. 3C shows first and second views 302c, 302c′ of the third GUI 300c generated by the VAD App, in accordance with some embodiments of the present specification. The first view 302c shows the VAD App has sensed that at least one of the head-mounted vision device 105 or the remote device is not connected to the computing device 110 and therefore displays a message 304c prompting to enable the connection while also displaying a visual graphical element 308c indicating that at least one of the head-mounted vision device 105 or the remote device is not connected to the computing device 110. The second view 302c′ shows the VAD App sensing that the head-mounted vision device 105 and the remote device are in data communication with the computing device 110 and hence displays a visual graphical element 310c indicating that the head-mounted vision device 105 and the remote device are in data communication with the computing device 110. Also, a visual graphical element 306c (titled “start order”) is enabled for actuation in order to initiate the scheduled eye exam order for the patient.

At step 210, the VAD App begins providing, sequentially, one or more content or agenda associated with the eye exam order to the patient through the head-mounted vision device 105 by concurrently generating data indicative of a fourth GUI 300d that has a plurality of views some of which are accessible to a technician or clinical staff person on the computing device 110 whereas others are accessible to the patient for viewing through the head-mounted vision device 105. In some embodiments, the eye exam order has a predefined sequential series of content or agenda (to be provided to the patient) corresponding to the patient's eye ailment and, hence, the eye exam, diagnostic or test.

FIG. 3D shows first view 352d, second view 354d, third view 356d, fourth view 358d, and fifth view 360d of the fourth GUI 300d generated by the VAD App, in accordance with some embodiments of the present specification. As shown in FIG. 3D, each of the first view 352d, second view 354d, third view 356d, fourth view 358d, and fifth view 360d, has a first portion or area 304d that includes a menu 306d of the predefined sequential series of content or agenda corresponding to the patient's eye ailment and, hence, the eye exam, diagnostic or test. The first portion or area 304d also displays the patient's name.

Each of the first view 352d, second view 354d, third view 356d, fourth view 358d, and fifth view 360d has a second portion or area 308d that includes a toggle-enabled visual graphical element having a first toggle position 310d and a second toggle position 310d′. Actuating the first toggle position 310d causes each of the first, second, third, fourth and fifth views 352d, 354d, 356d, 358d, 360d to be configured for viewing by the technician or clinical staff person on the computing device 110. Actuating the second toggle position 310d′ causes each of the first view 352d, second view 354d, third view 356d, fourth view 358d, and fifth view 360d to be configured for viewing by the patient on the head-mounted vision device 105. In some embodiments, the first and second toggle positions 310d, 310d′ are both enabled for actuating by the technician or clinical staff person. In some embodiments, the first toggle position 310d is disabled (for actuating) for the patient. In some embodiments, the second toggle position 310d′ is disabled (for actuating) for the technician or clinical staff person. It should be appreciated that, in some embodiments, the technician or clinical staff person is enabled to view a status of progress of the predefined sequential series of content or agenda by actuating the first toggle position 310d.

In some embodiments, the second portion or area 308d displays data or content corresponding to an agenda, from the menu 306d, that is in progress for the patient. The first view 352d displays the data or content in the context of or relevant to the technician or clinical staff person whereas the second view 354d displays the data or content in the context of or relevant to the patient. For example, for a calibration agenda 312d (from the menu 306d) the first view 352d displays (in the second portion or area 308d) a message that the patient is currently calibrating the head-mounted vision device 105 whereas the second view 354d displays (in the second portion or area 308d) an outline of the head-mounted vision device 105 with calibration in progress. The eye exam can be paused by actuating a visual graphical element 314d. The fourth GUI 300d includes another visual graphical element 316d (titled “step away mode”).

Thus, in some embodiments, as the patient progresses through his eye exam order, first view 352d and second view 354d of the fourth GUI 300d are generated, one for the technician or clinical staff person and the other for the patient. Also, as the patient progresses through the predefined sequential series of content or agenda (from the menu 306d), the first GUI 300a screen is also concurrently updated to reflect the patient's progress status on the eye exam.

As shown, in some embodiments, the predefined sequential series of content or agenda in the menu 306d has the calibration agenda 312d followed by a VFT (visual field test) practice test 315d. Conventionally, patients are not instructed to do a practice test because it takes a technician's or clinical staff person's time. It should be appreciated that, in the context of the present specification, the practice test 315d is important and patients take at least one “practice” test to become proficient at identifying and tracking stimuli related to various eye exams. Practice tests tend to have a great deal of noise or skewed data or sensitivity. The methods and systems of the present specification enable such a practice test to be delivered since the method 200 is patient driven.

In some embodiments, the predefined sequential series of content or agenda includes one or more educational content including text, audio and/or video. For example, as shown in the fourth GUI 300d, the menu 306d includes an education video agenda 318d (for example, “what is glaucoma?”). When the educational video agenda 318d commences for the patient, the second portion or area 308d displays data or content corresponding to the educational video agenda 318d, as shown in the third view 356d (with the first toggle position 310d disabled and the second toggle position 310d′ enabled), of FIG. 3D, of the fourth GUI 300d.

In some embodiments, the predefined sequential series of content or agenda includes at least one eye exam. For example, as shown in fourth and fifth views 358d, 360d of the fourth GUI 300d, the menu 306d includes an eye exam agenda 320d (for example, “24-2 VFT Standard”). When the eye exam agenda 320d commences for the patient, the second portion or area 308d displays data or content corresponding to the eye exam agenda 320d, as shown in the fourth and fifth views 358d, 360d, of FIG. 3D, of the fourth GUI 300d. The fourth view 358d displays data or content accessible to the patient on the head-mounted vision device 105 (with the first toggle position 310d disabled and the second toggle position 310d′ enabled) whereas the fifth view 360d displays data or content accessible to the technician or clinical staff person on the computing device 110 (with the first toggle position 310d enabled and the second toggle position 310d′ disabled). As a non-limiting example, for the patient, the fourth view 358d displays an outline of the head-mounted vision device 105 with a stimulus being presented in the left eye window 322d. On the other hand, for the technician or clinical staff person, the fifth view 360d displays a battery of stimuli 324d that the patient has not seen, seen, untested, or other characteristics.

In various embodiments, the VAD App also enables the technician or clinical staff person to select and add one or more of a plurality of predefined sequential series of content or agenda to a patient's eye exam order. To enable this, the VAD App, in some embodiments, generates data indicative of a fifth GUI 300e that is available to the technician or clinical staff person for access on the computing device 110.

FIG. 3E shows the fifth GUI 300e generated by the VAD App, in accordance with some embodiments of the present specification. As shown, the fifth GUI 300e includes first visual graphical element 302e, second visual graphical element 302e′, and third visual graphical element 302e″ for viewing and selecting/adding eye exam templates, eye exams and educational content, respectively, to the patient's eye exam order. Actuating the first visual graphical element 302e shows a first menu 304e of a plurality of eye exam templates. Each of the plurality of eye exam templates has an associated visual graphical element 304e′ (shown, for example, as a “+” indicator) which when actuated enables selecting and adding the corresponding eye exam template to the patient's eye exam order.

Actuating the second visual graphical element 302e′ shows a second menu 306e of a plurality of eye exams. Each of the plurality of eye exams has an associated visual graphical element 306e′ (shown, for example, as a “+” indicator) which when actuated enables selecting and adding the corresponding eye exam to the patient's eye exam order.

Actuating the third visual graphical element 302e″ shows a third menu 308e of a plurality of educational content. Each of the plurality of educational content has an associated visual graphical element 308e′ (shown, for example, as a “+” indicator) which when actuated enables selecting and adding the corresponding educational content to the patient's eye exam order. In some embodiments, the plurality of educational content may be grouped into one or more categories 308e″ for ease of access by the technician or clinical staff person.

In various embodiments, any subset of a plurality of eye exams, tests or diagnostics may be available for adding as agenda to a patient's eye exam order. The plurality of eye exams, tests or diagnostics may include tests such as, but not limited to, visual field tests (such as confrontation visual field tests, automated static perimetry tests, kinetic visual field tests, ERG, Amsler grid), visual acuity test (certain), visual refraction eye test, color blindness test, and cornea topography. In embodiments, the methods of the present specification focus on, but are not limited to automated static perimetry testing.

At step 212, after the patient has progressed through and completed the predefined sequential series of content or agenda in the eye exam order, the VAD App conveys completion of the eye exam order by generating data indicative of a sixth GUI 300f (FIG. 3F) that has a first view 302f accessible to the technician or clinical staff person on the computing device 110 and has a second view 302f accessible to the patient for viewing through the head-mounted vision device 105.

FIG. 3F shows the first and second views 302f, 302f of the sixth GUI 300f generated by the VAD App, in accordance with some embodiments of the present specification. Each of the first and second views 302f, 302f has the first portion or area 304d that includes the menu 306d of the predefined sequential series of content or agenda corresponding to the patient's eye ailment and, hence, the eye exam, diagnostic or test. Also, each of the first and second views 302f, 302f has the second portion or area 308d that includes the toggle-enabled visual graphical element having the first and second toggle positions 310d, 310d′.

In a non-limiting example, the first view 302f displays a message to the technician or clinical staff person (with the first toggle position 310d enabled) that the patient has completed the ordered eye exam. There may be other messages or prompts such as, for example, “return to your patient's room and view exam results to assist them with their headset and discharge or reorder a new test”. Additionally, a visual graphical element 304f is displayed which when actuated enables the technician or clinical staff person to view the results of the patient's eye exam.

The second view 302f displays a message to the patient (with the second toggle position 310d′ enabled) that the eye exam is complete. There may be other messages of prompts such as, for example, “When you are ready, take off your headset and wait for your technician. They have been notified and will be coming in shortly”. Additionally, the visual graphical element 304f is displayed which when actuated enables the patient to view the results of his eye exam.

### Change Analysis of a Plurality of Eye Exam Results

In accordance with some aspects of the present specification, the VAD App is configured to generate one or more GUIs to enable a clinician to review a patient's one or more eye exam results in a plurality of ways. In some embodiments, the clinician may access the eye exam results either using the computing device 110 that is used to deliver and drive the eye exam order on the head-mounted vision device 105. Alternatively or additionally, in some embodiments, the clinician may access the eye exam results using the clinician's computing device 130 by accessing the results from the database 125 via the network 120.

In some embodiments, the methods of the present specification allow for the generation of data indicative of a graphical user interface that enables a clinician (on a secondary computing device or the clinician's computing device) to select results of two or more eye exams associated with one or more eye exam orders delivered at different points in time to the patient, wherein the selected two or more eye exams may or may not correspond to a same eye exam order, wherein the second computing device is in data communication with the at least one server and the first computing device over the network, and wherein the at least one server stores said results of the two or more eye exams. Further, the methods allow for the generation of data indicative of yet another graphical user interface on the second computing for displaying an analysis of the results of the two or more eye exams. In embodiments, results of the two or more eye exams are generated by a) obtaining a first value at each first stimulus location in the first of the two or more eye exams, b) obtaining a second value at each second stimulus location in the second of the two or more eye exams, c) if the first stimulus location and the second stimulus location are equivalent, applying a function to generate a third value for said equivalent location and displaying said third value in a graphical user interface and d) if the first stimulus location has no equivalent location in the second of the two or more eye exams and/or if the second stimulus location has no equivalent location in the first of the two or more eye exams, displaying said first value and/or said second value in the graphical user interface. In embodiments, the analysis corresponds to displaying said results side-by-side for comparison. In other embodiments, the analysis corresponds to: determining a change in one or more parameters of said results; and displaying the change.

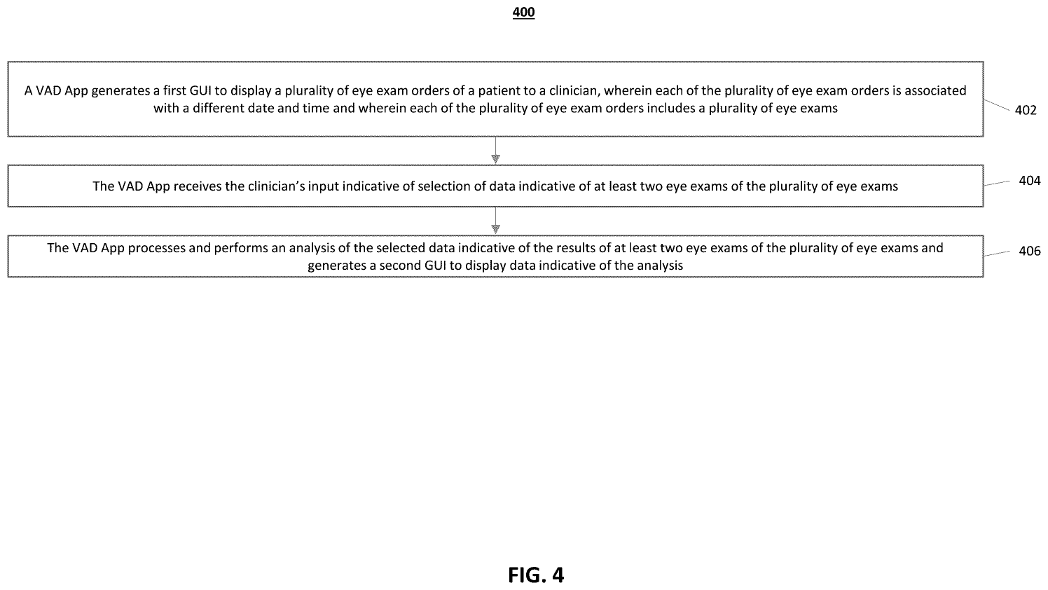

FIG. 4 is a flowchart of a plurality of exemplary steps of a method 400 of enabling a clinician to analyze and view results of a patient's one or more eye exams, in accordance with some embodiments of the present specification. In embodiments, the method 400 is implemented by the vision assist and diagnostic application (“VAD App”) on the computing device 110. However, the analysis of the results of the patient's one or more eye exams may also be viewed or accessed by the clinician remotely using the clinician's computing device 130. In embodiments, the one or more eye exams may be conducted as part of a predefined sequential series of content or agenda of an eye exam order delivered to the patient at a point in time and/or as part of predefined sequential series of content or agenda of a plurality of eye exam orders delivered to the patient at different points in time.

Referring now to FIGS. 1, 4, 5A, 5B and 5C, at step 402, the VAD App generates a seventh GUI 500a to display a plurality of eye exam orders of the patient to the clinician, wherein each of the plurality of eye exam orders is associated with a different date and time and wherein each of the plurality of eye exam orders includes a plurality of eye exams.

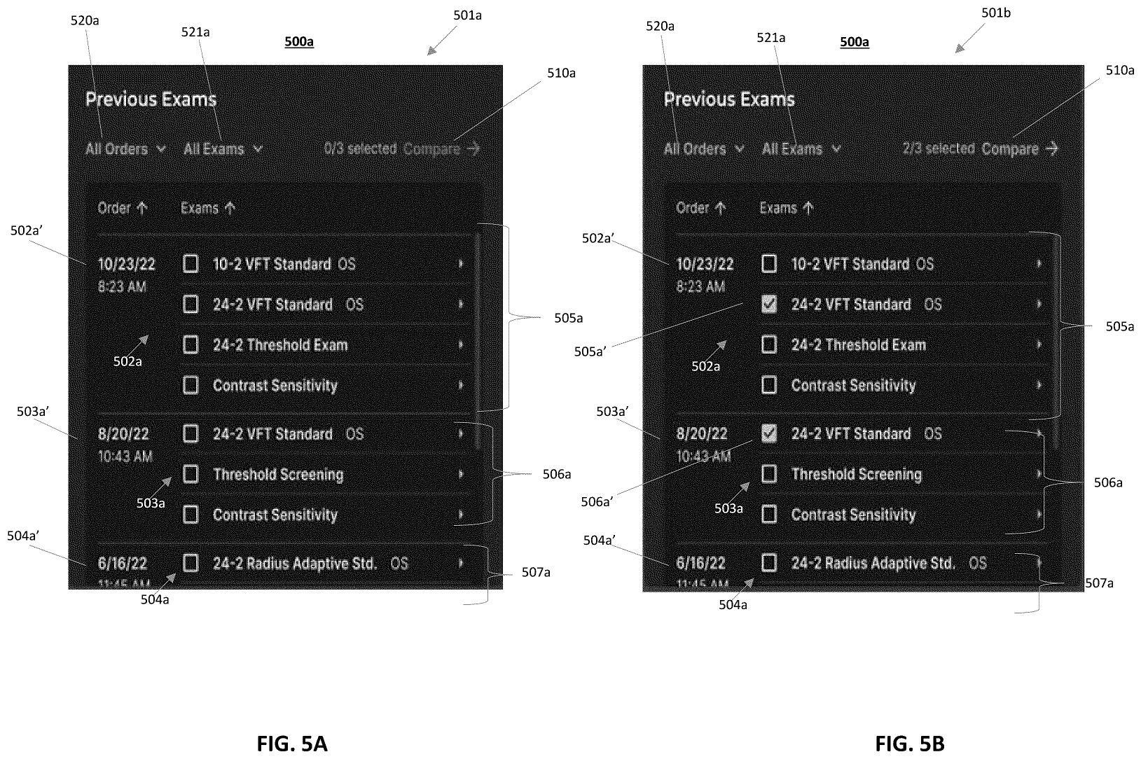

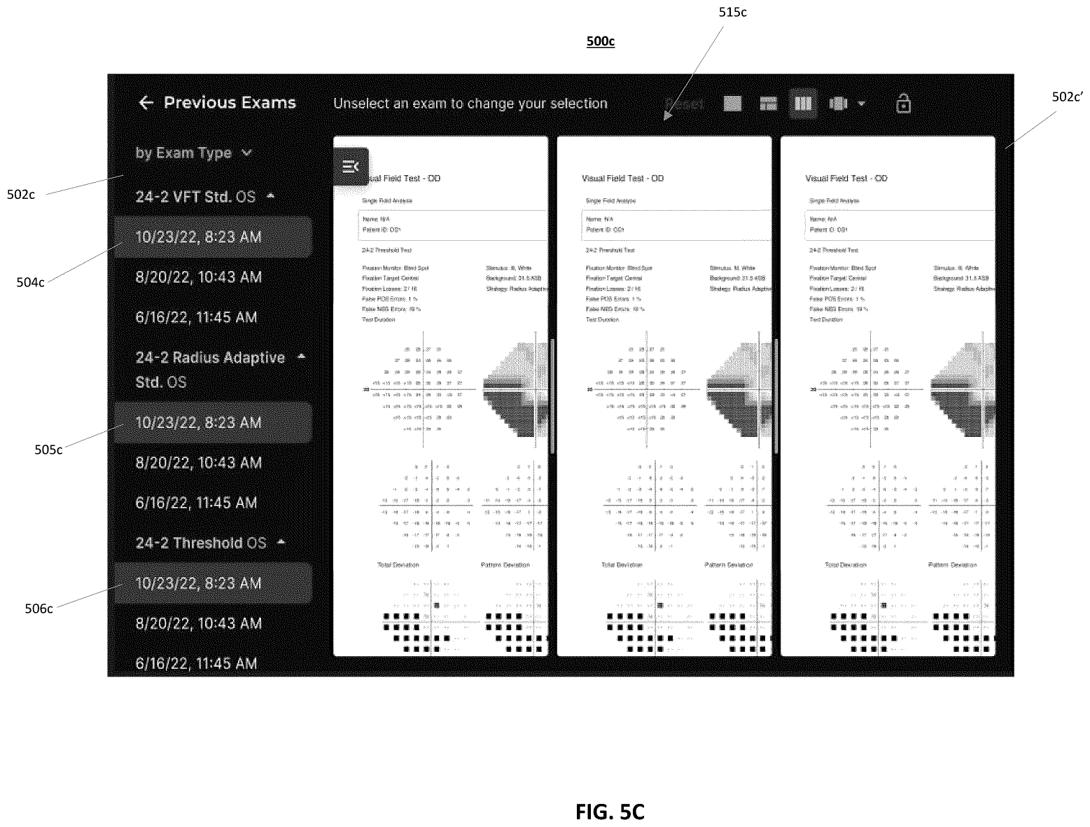

FIG. 5A shows a first view 501a of the seventh GUI 500a generated by the VAD App, in accordance with some embodiments of the present specification. As shown, the seventh GUI 500a displays eye exam orders 502a, 503a, 504a of the patient that are an exemplary subset of a plurality of eye exam orders. Each of the eye exam orders 502a, 503a, 504a has an associated date and time stamp 502a′, 503a′, 504a′. Thus, the eye exam orders 502a, 503a, 504a were delivered to the patient at different points in time and therefore represent previous or historical eye exam orders. A visual graphical element 520a is configured to enable the selection or filtering of the plurality of eye exam orders on the basis of an eye exam order while another visual graphical element 521a is configured to enable selection or filtering of the plurality of eye exam orders on the basis of a type of eye exam. As also shown, each of the plurality of eye exam orders 502a, 503a, 504a includes a plurality of eye exams 505a, 506a, 507a as part of the predefined sequential series of content or agenda of the eye exam order. Additionally, each of the plurality of eye exams 505a, 506a, 507a has an associated check-box for selection of the corresponding eye exam.

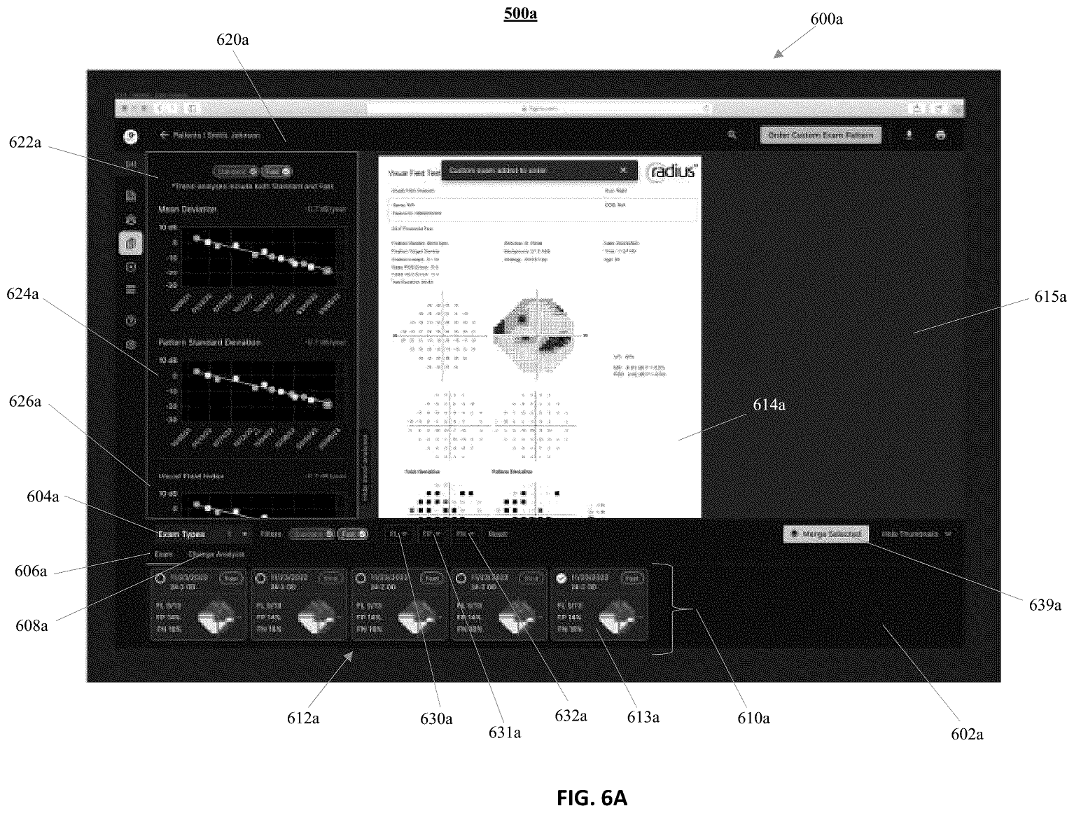

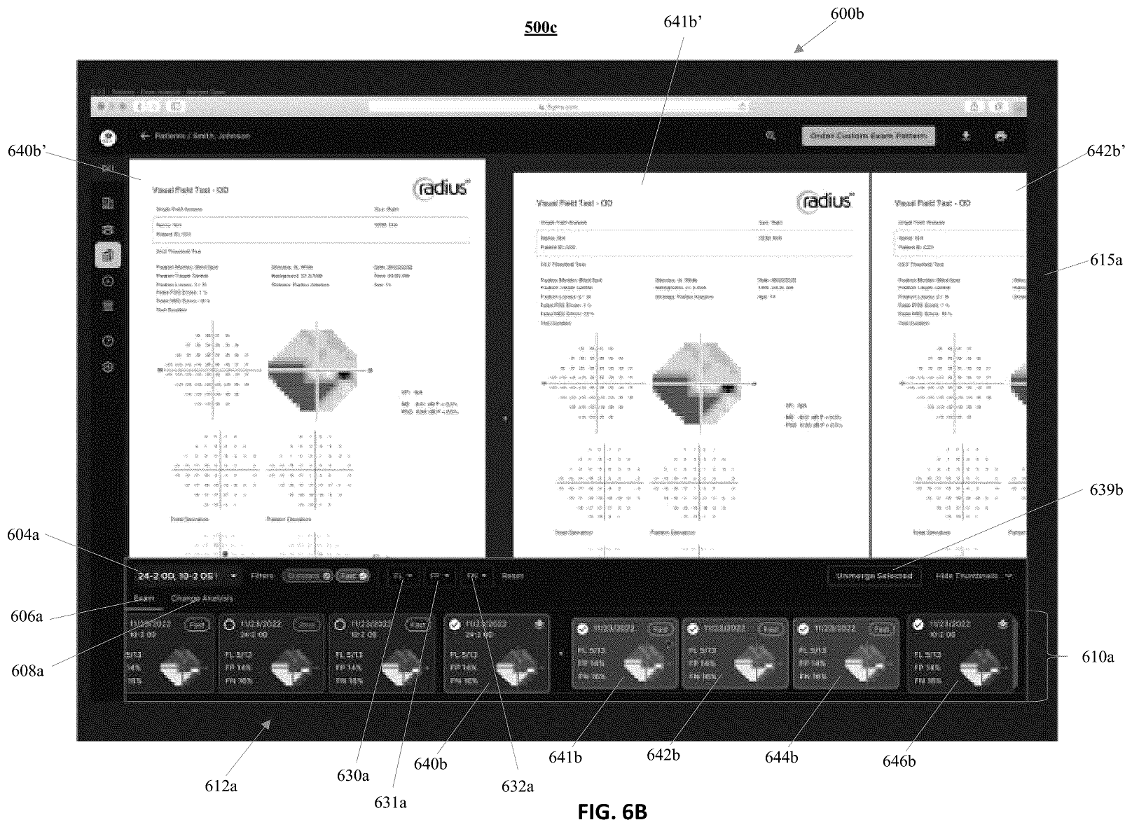

FIG. 6A is an alternate view or GUI 600a of the seventh GUI 500a generated by the VAD App, in accordance with alternate embodiments of the present specification. The alternate view or GUI 600a is configured such that it enables selection of one or more of a plurality of eye exam types provided to the patient at various dates and times. As shown, view 600a includes a bottom portion 602a having a drop-down box 604a configured to enable filtering a plurality of eye exam orders on the basis of an eye exam type. Positioned below the drop-down box 604a are a first visual graphical element 606a and a second visual graphical elements 608a. The first visual graphical element 606a, when actuated, is configured to enable visualization of one or more eye exams based on an eye exam type selected from the drop-down box 604a and related data, functionalities and features. The second visual graphical element 608a, when actuated, is configured to enable data, functionalities and features related to comparative or change analysis with reference to at least two eye exams of the patient.

Since the first visual graphical element 606a is shown as actuated in the figure, view 600a is configured for data, functionalities, and features related to an eye exam type selected from the drop-down box 604a. A horizontal tray 610a is configured to display a plurality of eye exams 612a, in visual miniature forms/icons, characterized by the eye exam type selected from the drop-down box 604a. In some embodiments, by default, the plurality of eye exams 612a are presented left to right sorted in an ascending order of oldest to earliest eye exam. However, the plurality of eye exams 612a may alternatively be sorted in a descending order (or any other order) to create the horizontal tray 610a. In embodiments, third visual graphical element 630a, fourth visual graphical element 631a, and fifth visual graphical element 632a are configured as drop-down boxes to enable the plurality of eye exams 612a to be filtered for metrics such as, for example, fixation loss, false positives, and false negatives, respectively. The metrics of fixation loss, false positives and false negatives are indicative of reliability of an eye exam. Consequently, the third visual graphical element 630a, fourth visual graphical element 631a, and fifth visual graphical element 632a may be manipulated to filter out unreliable eye exams. For example, the third visual graphical element 630a may be manipulated to filter out eye exams with fixation loss of higher than 30%.

Each of the plurality of eye exams 612a includes a radio button which when actuated is configured to indicate selection of the corresponding eye exam of the plurality of eye exams 612a. One or more eye exams may be selected from the tray 610a. An exemplary eye exam 613a is shown as selected from the plurality of eye exams 612a.

Upon selection of the eye exam 613a, corresponding test data 614a is displayed in a first window 615a. Also, a plurality of statistical data analysis is displayed in a second window 620a, concurrently with the test data 614a. The first window 615a and second window 620a are positioned adjacent to each other and above the tray 610a. In some embodiments, the plurality of statistical data analysis is derived from the collective test data corresponding to the plurality of eye exams 612a displayed in the tray 610. In some embodiments, the plurality of statistical data analysis is shown both numerically and graphically and includes a first statistical analysis 622a indicative of mean deviation, a second statistical analysis 624a indicative of pattern standard deviation, and a third statistical analysis 626a indicative of visual field index.

It should be appreciated that view 600a also includes a visual graphical element 639a (titled ‘merge selected’) which, when actuated, is configured to merge data indicative of at least two selected eye exams from the plurality of eye exams 612a.

At step 404, the VAD App receives the clinician's input indicative of selection of data indicative of at least two eye exams of the plurality of eye exams. In some embodiments, the VAD App allows the clinician to select any number of the plurality of eye exams. In some embodiments, the VAD App allows the clinician to select a predefined, yet customizable, maximum number of the plurality of eye exams.

FIG. 5B shows a second view 501b of the seventh GUI 500a, where the clinician has selected first and second eye exams 505a′, 506a′. As shown in FIGS. 5A and 5B, the seventh GUI 500a also includes a visual graphical element 510a which when actuated by the clinician causes the VAD App to generate a comparative analysis of the selected at least two eye exams of the plurality of eye exams.

At step 406, the VAD App is configured to process and perform an analysis of the selected data indicative of the results of at least two eye exams of the plurality of eye exams and generates an eighth GUI 500c (FIG. 5C) to display data indicative of the analysis. In some embodiments, the analysis may be comparative in nature where the data indicative of the at least two eye exams is displayed to the user side-by-side for ease of comparison. In some embodiments, the analysis may be reflective of a change in the patient's visual acuity or eye health over a period of time with reference to the at least two eye exams.

FIG. 5C shows the eighth GUI 500c generated by the VAD App, in accordance with some embodiments of the present specification. As shown, the eighth GUI 500c has a first portion or area 502c and a second portion or area 502c′. The first portion or area 502c displays a list of historical eye exams conducted for the patient. As a non-limiting example, first, second and third eye exams 504c, 505c, 506c are shown highlighted indicative of the fact that these eye exams (that may have been conducted at different points in time) were selected by the clinician (at step 404) for further analysis and display.

The second portion or area 502c′ displays data indicative of an analysis performed by the VAD App on the first, second and third eye exams 504c, 505c, 506c.

In a first non-limiting example, as shown, the second portion or area 502c′ displays first, second, and third data—indicative of the results of the first, second and third eye exams 504c, 505c, 506c—in a comparative mode 515c such that the first, second and third data are displayed side-by-side for ease of comparison by the clinician.

In a second non-limiting example, the second portion of area 502c′ displays an analysis indicative of a change (improvement, deterioration) or no appreciable change in one or more parameters related to the patient's eye health over a period of time with reference to data points (that is, results) indicative an eye exam. To enable the change analysis, in some embodiments, the VAD App merges data indicative of at least one eye exam, selected by the clinician, in order to generate merged data and treats the merged data as if it was generated from a single eye exam. Thus, the clinician is enabled to specify which of the eye exam data, for merging, corresponds to baseline and therefore which of the eye exam data (here, the data is referred to as a sensitivity estimate), for merging, corresponds to post-baseline. For example, there may be a total of ‘N’ visual field tests performed for a given eye over time. In embodiments, only one visual field test is administered per visit, testing K locations at which sensitivity estimates are generated. In an embodiment, the sensitivity estimate is generated based on a “visual threshold” at each location, meaning the brightness or intensity at which the patient will view or observe the stimulus 50% of the time at that location. A first subset of measurements K_{B} (of the selected K sensitivity estimates from the N number of tests) may be selected by the clinician, from at least one exam, to be merged into (or considered as in the case of a single measurement) into a single baseline visual field sensitivity estimate, while a second subset of measurements K_{P} (of the selected K sensitivity estimates from the N number of tests) could be selected, from at least one eye exams, to be merged into a single post-baseline visual field sensitivity estimate. In embodiments, it is desired that every visual field sensitivity estimate within the second subset K_{P} comes after (as in the time point that visual field was measured) every visual field sensitivity estimate within the first subset K_{B} since baseline comes before post-baseline. For both baseline and post-baseline at least one visual field sensitivity estimate is required. The clinician, in embodiments, chooses at least one K value for baseline generation. In embodiments, a clinician may choose to merge only visual fields that are reliable. In an embodiment, but not limited to such embodiment, where a 24-2 test is employed, a patient's visual field is tested at 54 locations, generating 54 different sensitivity estimates (K=54). In an embodiment, but not limited to such embodiment, where a 10-2 test is employed, a patient's visual field is tested at 68 locations, generating 68 different sensitivity estimates (K=68).

It should be appreciated that the term baseline is indicative of a point prior to intervention, a starting value (i.e., first time the patient visits the clinician), or a new baseline post-intervention. The change analysis enables the clinician to custom select baseline and post-baseline eye exam data and then review the difference between the merged visual field test results. Each visual field test consists of many test locations (for example, the 24-2 test pattern has 54 test locations), so the difference between baseline and post-baseline (mathematically, post-baseline minus baseline) is displayed at every test location. This allows the clinician to see how sensitivity changed at each test location from baseline to post-baseline. In some embodiments, for change analysis it is desired that the visual fields, from the first eye exam to each subsequent eye exam, have the same test pattern (for example, 24-2 test pattern) and sensitivity is compared at each test location baseline versus post-baseline.

FIG. 6B shows an alternate view 600b of the eighth GUI 500c generated by the VAD App, in accordance with alternate embodiments of the present specification. Referring now to FIGS. 6A and 6B, view 600b shows visual miniature forms/icons indicative of eye exams 640b (merged data), 641b, 642b, 644b and 646b selected from the horizontal tray 610a. Consequently, the first window 615a displays data (such as, for example, data 640b′ (merged data), 641b′, 642b′, which in FIG. 6B comprise only a portion of test data corresponding to the selected eye exams) corresponding to the selected eye exams 641b, 642b, 644b and 646b, respectively. In accordance with some embodiments, the data 640b and 640b′ is indicative of merged data generated by the VAD App by merging data indicative of the selected eye exams 641b, 642b, 644b and 646b. In some embodiments, the merged data (such as, merged data 640b′) is generated upon selecting at least two eye exams and thereafter actuating the visual graphical element 639a (titled ‘merge selected’) shown in view 600a of FIG. 6A. Once actuated, the visual graphical element 639a toggles to a visual graphical element 639b (titled ‘unmerge selected’) to unmerge data of selected at least two eye exams. Thus, the visual graphical elements 639a, 639b toggle between ‘merge selected’ functionality of view 600a and ‘unmerge selected’ functionality of view 600b, respectively.

In embodiments, when a test is administered to a patient, for each test point in an eye exam (for example, the first eye exam and second eye exam), a small spot of light is presented as stimulus to the patient at different light intensities. The patient responds by saying ‘yes’ if he is able to see the light and by saying ‘no” if he is unable to see the light. Thereafter, a graph is generated by plotting the light intensities on an X-axis and by plotting a value of 0 or 1 (for ‘yes’ or ‘no’) on the Y-axis for each of the light intensities. The process is repeated for all test points in each of the first eye exams and second eye exams. Thereafter, a predefined psychometric function, such as a Weibull, cumulative normal, logistic, or any other sigmoidal function is fitted to the test points in an eye exam such that a midpoint of the fitted function corresponds to the patient's sensitivity.

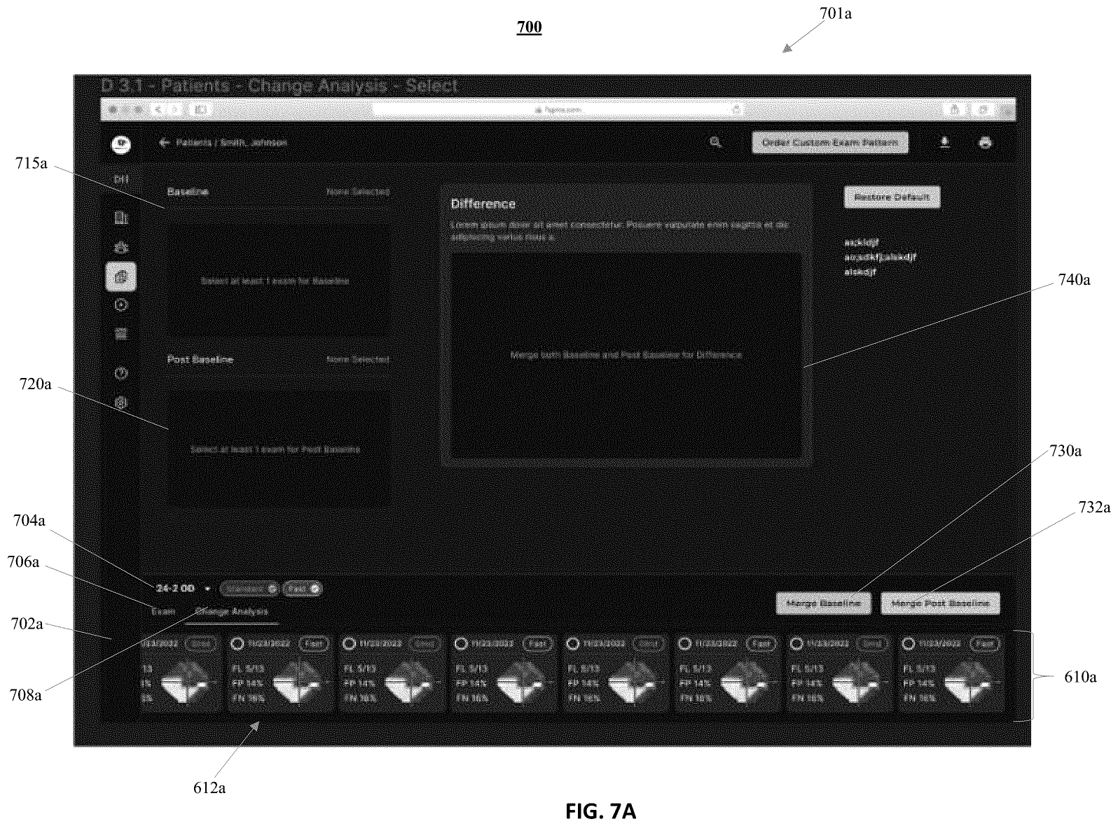

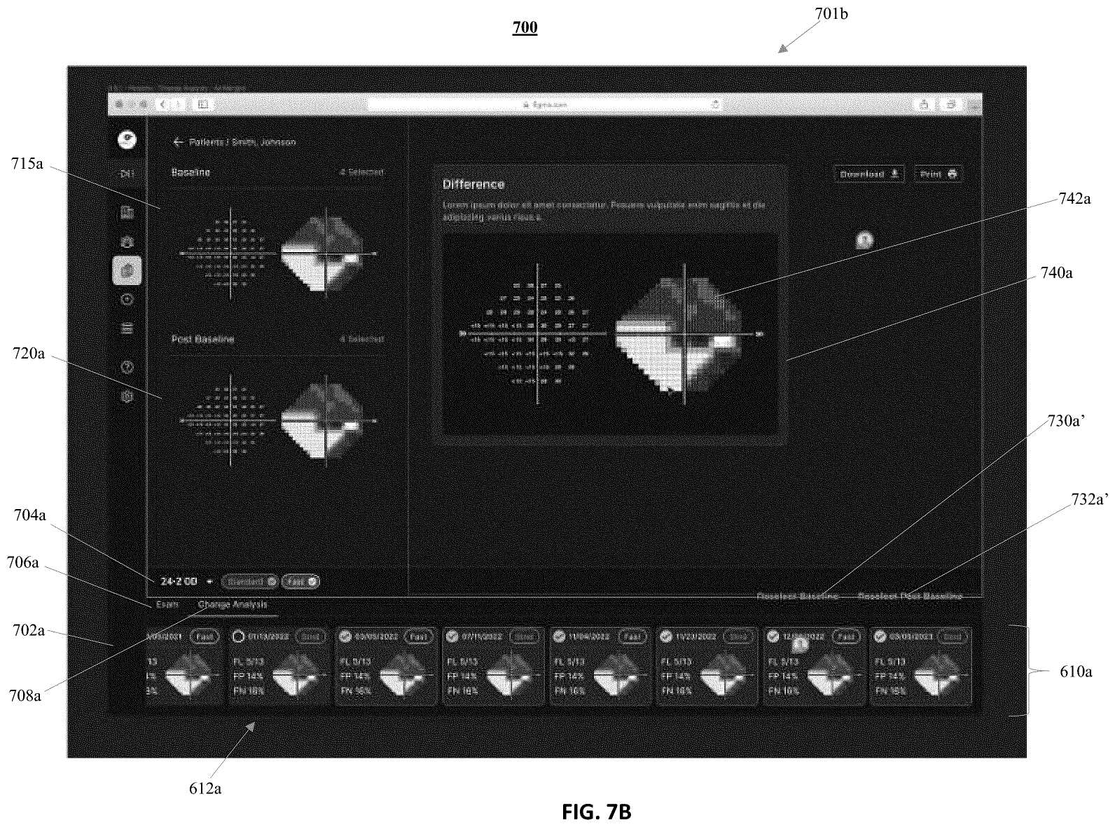

FIGS. 7A and 7B respectively show first view 701a and second view 701b of a GUI 700 generated by the VAD App that is displayed and configured to enable a clinical staff person to select baseline and post-baseline eye exam data for merging, in accordance with alternate embodiments of the present specification. As shown, the views 701a, 701b include a bottom portion 702a having a drop-down box 704a configured to enable filtering a plurality of eye exam orders on the basis of eye exam types. Positioned below the drop-down box 704a are a first visual graphical element 706a and a second visual graphical elements 708a. The first visual graphical element 706a, when actuated, is configured to enable visualization of one or more eye exams based on an eye exam type selected from the drop-down box 704a and related data, functionalities and features. The second visual graphical element 708a, when actuated, is configured to enable data, functionalities and features related to change analysis with reference to at least two eye exams of the patient.

Since the second visual graphical element 708a is shown as actuated, the view 701a is configured for data, functionalities and features related to change analysis with reference to two or more eye exams related to an eye exam type selected from the drop-down box 704a. A horizontal tray 710a is configured to display a plurality of eye exams 712a, in visual miniature forms/icons, characterized by the eye exam type selected from the drop-down box 704a.

To select baseline eye exam data for merging, at least two eye exams are selected from the plurality of eye exams 712a and, subsequently, a first visual graphical element 730a (titled ‘merge baseline’ and shown in view 701a) is actuated. This causes the selected baseline eye exam data to be displayed in a first window 715a. Thereafter, the first visual graphical element 730a toggles to a third visual graphical element 730a′ (titled ‘reselect baseline’ and shown in view 701b) to allow reselection of baseline eye exam data. Similarly, to select post-baseline eye exam data for merging, at least two eye exams are selected from the plurality of eye exams 712a and, subsequently, a second visual graphical element 732a (titled ‘merge post-baseline’ and shown in view 701a) is actuated. This causes the selected post-baseline eye exam data to be displayed in a second window 720a. Thereafter, the second visual graphical element 732a toggles to a fourth visual graphical element 732a′ (titled ‘reselect post-baseline’ and shown in view 701b). In some embodiments, data corresponding to only similar or like eye exams can be merged.

Once the baseline and post-baseline eye exam data is selected, a third window 740a displays merged eye exam data 742a, indicative of change analysis, based on the selected baseline and post-baseline eye exam data. As an example, ‘M’ eye exam points or locations correspond to ‘M’ numbers which show increase or decrease in the merged eye exam data 742a visually presented in the third window 740a. In some embodiments, the visually presented merged eye exam data 742a uses a first color indicative of increase and a second color indicative of decrease of the numbers corresponding to each of the ‘M’ eye exam locations.

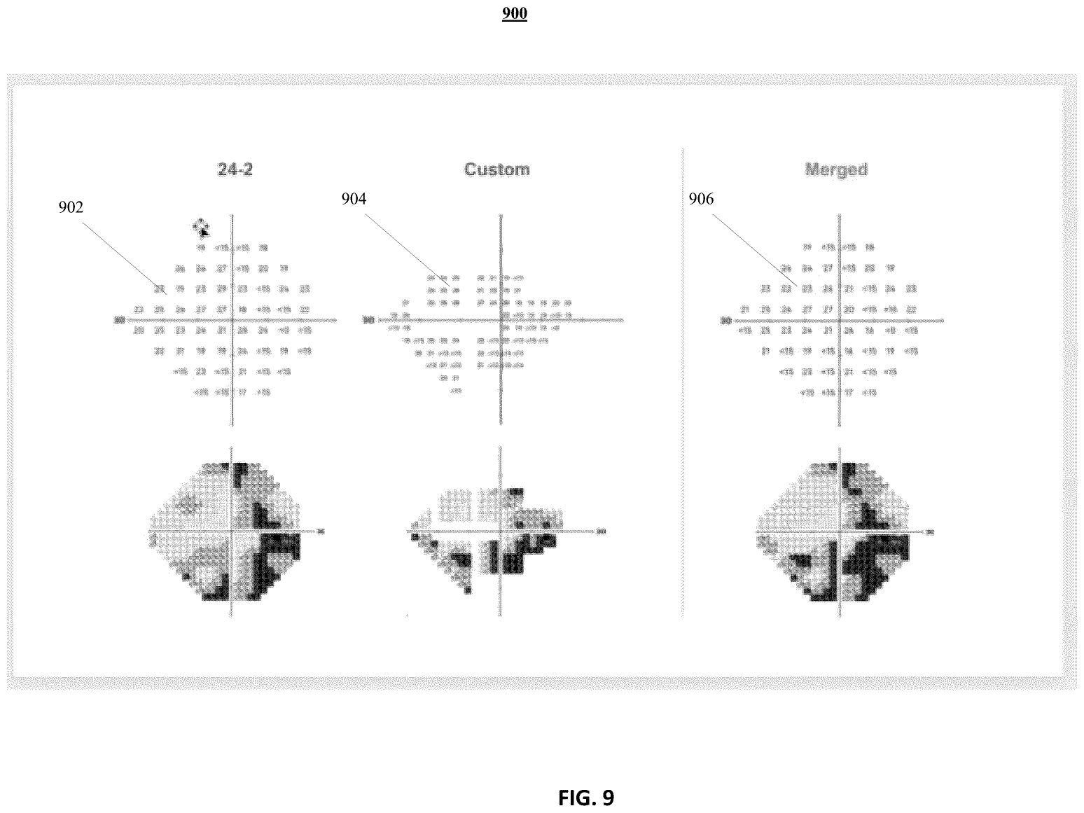

In some embodiments, a standard eye exam (such as, for example, a 24-2 visual test) may be performed initially, followed by a custom eye exam (such as, by using the functionalities enabled by the GUI 800 of FIG. 8) with custom or new locations and density based on the initial standard eye exam. Thereafter, first data from the standard eye exam may be merged with second data from the custom eye exam. As shown in GUI 900 of FIG. 9, first data 902 indicative of a standard eye exam (such as, for example, a 24-2 visual test) is merged with second data 904 indicative of a custom eye exam in order for the VAD App to generate third data 906 indicative of merged data.

It should be noted that conventional guided progression analysis (treats the first two as baseline and then the next three as post-baseline) averages over each eye exam point or location but does not show the actual averaged data at each eye exam point or location and only works with the same eye exam points or locations. In contrast, the VAD App merges each eye exam point or location and shows the actual average data at each eye exam point or location. This visual demonstration is critical because it shows the spatial correlation between various eye exam points or locations that is clinically meaningful and that is not typically shown. In showing the different sensitivity estimates at each location for the visual field tests that are administered over time, a clinician is able to remove and skewed or outlier data in order to arrive at a threshold that is more accurate for the patient. It should be noted that in various embodiments, it may be possible to compare and merge the results of different visual field test patterns (for example 24-2 and 10-2) by interpolating or extrapolating at a set of predetermined locations in the visual field.

The above examples are merely illustrative of the many applications of the systems and methods of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A method of delivering an eye exam order to a patient using a head-mounted vision device configured to be positioned on the patient's head, wherein the head-mounted vision device includes a display in electrical and data communication with a first computing device that is in data communication with at least one server over a network, and wherein the first computing device includes a non-transient memory in data communication with at least one processor and adapted to store programmatic instructions that, when executed, execute said method, the method comprising:
generating, for display on the first computing device, data indicative of a first graphical user interface to enable the patient to self-check-in for receiving the eye exam order;
positioning the head-mounted vision device on the patient's head;
generating, for display on the head-mounted device, data indicative of a first view of a second graphical user interface to deliver a predefined plurality of content items to the patient; and
generating, for display on the first computing device, data indicative of a second view of the second graphical user interface to display progress status of the predefined plurality of content items to a clinical staff person.

2. The method of claim 1, wherein the first graphical user interface is configured to display the patient's information and eye exam order amongst a plurality of patients' information and associated eye exam orders.

3. The method of claim 2, wherein the patient, using the first computing device, actuates the eye exam order to self-check-in.

4. The method of claim 1, wherein the second graphical user interface includes a visual graphical element having first and second toggle positions.

5. The method of claim 4, wherein enabling the first toggle position causes the first view to be displayed, and wherein enabling the second toggle position causes the second view to be displayed.

6. The method of claim 1, wherein the predefined plurality of content items comprises one or more eye exams and one or more educational content related to one or more eye ailments.

7. The method of claim 1, further comprising:
generating, for display on the first computing device, data indicative of a third graphical user interface to enable the clinical staff person to add at least one of: one or more eye exam templates, one or more eye exams and one or more educational content to the eye exam order.

8. The method of claim 1, further comprising:
generating, for display on a second computing device, data indicative of a fourth graphical user interface to enable a clinician to select results of two or more eye exams associated with one or more eye exam orders delivered at different points in time to the patient, wherein the selected two or more eye exams may or may not correspond to a same eye exam order, wherein the second computing device is in data communication with the at least one server and the first computing device over the network, and wherein the at least one server stores said results of the two or more eye exams; and
generating, for display on the second computing device, data indicative of a fifth graphical user interface configured to display an analysis of said results of the two or more eye exams.

9. The method of claim 8, wherein said results of the two or more eye exams are generated by a) obtaining a first value at each first stimulus location in the first of the two or more eye exams, b) obtaining a second value at each second stimulus location in the second of the two or more eye exams, c) if the first stimulus location and the second stimulus location are equivalent, applying a function to generate a third value for said equivalent location and displaying said third value in the fifth graphical user interface and d) if the first stimulus location has no equivalent location in the second of the two or more eye exams and/or if the second stimulus location has no equivalent location in the first of the two or more eye exams, displaying said first value and/or said second value in the fifth graphical user interface.

10. The method of claim 8, wherein the fifth graphical user interface is configured to display the analysis of said results of the two or more eye exams side-by-side for comparison.

11. The method of claim 8, wherein the analysis corresponds to determining a change in one or more parameters of said results and wherein the fifth graphical user interface is configured to display the change.

12. A computer readable program adapted to deliver an eye exam order to a patient using a head-mounted vision device positioned on the patient's head, wherein the head-mounted vision device includes a display in electrical and data communication with a first computing device that is in data communication with at least one server over a network, wherein the first computing device includes at least one processor in data communication with a non-transient memory that stores the computer readable program, and wherein the computer readable program comprises a plurality of programmatic instructions that, when executed by the at least one processor:
generates, for display on the first computing device, data indicative of a first graphical user interface to enable the patient to self-check-in for receiving the eye exam order;
generates, for display on the head-mounted device, data indicative of a first view of a second graphical user interface to deliver a predefined plurality of content items to the patient; and
generates, for display on the first computing device, data indicative of a second view of the second graphical user interface to display progress status of the predefined plurality of content items to a clinical staff person.

13. The computer readable program of claim 12, wherein the first graphical user interface is configured to display the patient's information and eye exam order amongst a plurality of patients' information and associated eye exam orders.

14. The computer readable program of claim 13, wherein the patient, using the first computing device, actuates the eye exam order to self-check-in.

15. The computer readable program of claim 12, wherein the second graphical user interface includes a visual graphical element having first and second toggle positions.

16. The computer readable program of claim 15, wherein enabling the first toggle position causes the first view to be displayed, and wherein enabling the second toggle position causes the second view to be displayed.

17. The computer readable program of claim 12, wherein the predefined plurality of content items comprises one or more eye exams and one or more educational content related to one or more eye ailments.

18. The computer readable program of claim 12, further comprising:
generating, for display on the first computing device, data indicative of a third graphical user interface to enable the clinical staff person to add at least one of: one or more eye exam templates, one or more eye exams and one or more educational content to the eye exam order.

19. The computer readable program of claim 12, further comprising:
generating, for display on a second computing device, data indicative of a fourth graphical user interface to enable a clinician to select results of at least two eye exams associated with one or more eye exam orders delivered at different points in time to the patient, wherein the selected at least two eye exams may or may not correspond to a same eye exam order, wherein the second computing device is in data communication with the at least one server and the first computing device over the network, and wherein the at least one server stores said results of the at least two eye exams; and
generating, for display on the second computing device, data indicative of a fifth graphical user interface displaying an analysis of said results of the at least two eye exams.

20. The computer readable program of claim 19, wherein the fifth graphical user interface is configured to display the analysis of said results of the two or more eye exams side-by-side for comparison.
